(19) United States
(12) Patent Application Publication
(10) Pub. No.: US 2011/0137111 A1
(43) Pub. Date: Jun. 9, 2011

(54) SYSTEMS METHODS AND APPARATUSES FOR REHABILITATION OF AUDITORY SYSTEM DISORDERS

(75) Inventors: Peter John Hanley, New South Wales (AU); Robert Henry Frater, New South Wales (AU); Paul Benjamin Davis, Fort Lauderdale, FL (US)

(73) Assignee: Neuromonics Pty Ltd, Chatswood New South Wales (AU)

(21) Appl. No.: 12/736,506

(22) PCT Filed: Apr. 9, 2009
(86) PCT No.: PCT/AU2009/000451

§ 371 Date: Feb. 18, 2011

Related U.S. Application Data
(60) Provisional application No. 61/071,254, filed on Apr. 18, 2008.

(51) Int. Cl. A61M 21/02

(52) U.S. Cl. 600/28

(57) ABSTRACT

Systems, methods and apparatuses for auditory system disorder rehabilitation by providing a stimulus to the auditory system of an individual experiencing an auditory disorder, tinnitus, conditions of reduced tolerance of loud sounds, or combinations thereof comprising, for example, a playback device and a method and apparatus for modifying an audio signal. The methods may include allowing a user to select an audio source (for example, a music track or the audio output of a television program) and modifying the audio with a spectral modified signal to create a spectrally modified audio. The spectrally modified audio can then be output by the playback device or other means for listening by the user.

### CROSS-REFERENCE TO RELATED APPLICATIONS

This Application claim priority to U.S. Provisional Application No. 61/071,254, filed Apr. 18, 2008. This application is also related to U.S. application Ser. No. 11/921,500, filed on Dec. 4, 2007, which is a U.S. National Stage application of International Application No. PCT/AU2006/000777, filed on Jun. 7, 2006, which claims the benefit of U.S. Provisional Application No. 60/689,088; and of U.S. application Ser. No. 10/727,036, filed on Dec. 4, 2003, which is a continuation-in-part of U.S. application Ser. No. 09/936,687, now U.S. Pat. No. 6,682,472, filed on Sep. 17, 2001 which is a U.S. National Stage application of International Application No. PCT/AU00/00207, filed on Mar. 17, 2000, which claims the benefit of Australian Provisional Application No. PP9275, filed on Mar. 17, 1999. Each of these applications, in their entirety, is herein incorporated by reference.

### BACKGROUND

1. Field of the Disclosure

The present disclosure relates to systems, methods and apparatuses for rehabilitation of auditory system disorders. More particularly, but not exclusively, the present disclosure relates to systems, methods and apparatuses for auditory system rehabilitation by providing a stimulus to the auditory system of an individual experiencing tinnitus or conditions of reduced tolerance of loud sounds.

2. Description of Related Art

A large percentage of the population experiences some form of an auditory system disorder. For many people, the disorder can be extremely disturbing and can, in some instances, lead to additional disorders. Tinnitus and conditions of reduced sound tolerance, including hyperacusis, are examples of often disturbing types of auditory system disorders. Hyperacusis involves a severe intolerance of moderately loud external noises. Tinnitus is commonly associated with hyperacusis and people with tinnitus perceive sounds that are not present in the external environment and/or that other people cannot generally hear. These sounds can include, for example, ringing in the ears, beating, pounding, buzzing and humming background sounds, roaring, or whistling noises in the ears.

Tinnitus can have a negative impact on work, family and social life and can lead to an inability to relax and disturbance of concentration and sleep patterns. Tinnitus can be caused by hearing loss resulting from exposure to loud noises, certain types of drugs & medication, or middle ear infections. In some instances, tinnitus arises from a condition that requires medical or surgical intervention.

It is estimated that a large proportion of the population (around 15%), experience some degree of tinnitus. For a small proportion, estimated around 1-2% of the general population, a secondary reaction involving the auditory cortex, the brain stem, the limbic and autonomic systems leads to significant distress and disturbance. This reaction appears to involve neural rewiring as recently demonstrated with MRI and PET brain scanning.

A portion of the people who suffer from tinnitus can be highly disturbed by it. Continuous perception of tinnitus can lead to insomnia, an inability to relax, anxiety, depression, and even suicide in extreme cases. Hyperacusis can generally occur in association with tinnitus, and is thought to share the same underlying causes. Thus, every reference to tinnitus in this document should be construed as including the phenomena of hyperacusis or other types of reduced tolerance of loud sounds.

There are few effective treatment options available for tinnitus sufferers, with the vast majority often being advised that “you'll have to learn to live with it”. Most patients find that they can far more readily ignore an external sound than their tinnitus. Some of the previous methods have used hearing aid-style devices that produce a band of noise in an attempt to totally “mask” or cover up the perception of the tinnitus. Such masking can give a sense of relief and control over the tinnitus in a portion of patients for whom the devices allow them to cover up their tinnitus perception. However, for many patients, the presence of hearing loss for external sounds in the frequency region where the tinnitus is prominent often means that the masking noise needs to be unpleasantly loud before the tinnitus can be masked to an appreciable degree, and the noise is often judged to be not much better than the tinnitus itself. These devices have not been shown to provide long-term effect and the cost and aesthetic considerations limit the proportion of sufferers for whom this is a viable measure.

Other reported devices attempt to achieve masking using devices that deliver vibrations (U.S. Pat. No. 5,692,056), pulsed ultrasonic stimulation (U.S. Pat. No. 6,394,969), or radio frequency waves (e.g., “Theraband™”) to the patient, while other reported devices (e.g., U.S. Pat. No. 5,697,975) seek to achieve stimulation through direct electrical discharge to the brain or provide relief by delivering an acoustic stimulus at a level that is inaudible to the patient (WO 01/70110).

Over the past decade, a further understanding of the neurophysiological processes underlying tinnitus has been published, emphasizing the role of the neural pathways in the emergence of distressing tinnitus and the possibility of using this neural plasticity to retrain its perception. This retraining approach has been dubbed “Tinnitus Retraining Therapy” or TRT. In this technique, patients are often given intensive counseling, and sometimes use noise generators at a volume that does not completely mask the tinnitus. Long term reductions in tinnitus disturbance have been achieved in some patients, but it is usual for this process to take at least 18 months of therapy before any substantial benefit occurs. TRT also offers little immediate sense of relief from the tinnitus, nor relief from the associated sleep disturbance and inability to relax.

Another known method was the “Silentia Set” developed by Starkey Corp., which is a pair of hearing aid devices which wirelessly receive signals from a stereo system via an induction loop under a pillow at bedtime. Recording of high frequency noise bands (“water sounds”), babble noise, traffic sounds and music have been used to mask tinnitus using this system, however the cost of the Silentia Set make it prohibitive for many sufferers.

Other known audiotherapeutic techniques using music are the Tomatis Method developed by Alfred A. Tomatis, and Auditory Integration Training. While neither method is designed for the treatment of tinnitus, the two techniques modify music for the treatment of auditory disorders. The Tomatis Method employs an “Electronic Ear” developed by Alfred Tomatis, (U.S. Pat. No. 4,021,611). It has its origins from an outdated model of how the auditory system works, and has been widely debunked by audiological organizations. Auditory Integration Training is based on the Tomatis Method, but presents the music at extremely loud levels, that may result in hearing damage, and importation of devices using this technique have been banned by the American Food and Drug Administration.

The present inventors have observed that it would be desirable to provide more effective rehabilitation techniques for people suffering from auditory system disorders based in part on a better understanding of the neural processes underlying the auditory system disorders.

### BRIEF DESCRIPTION OF THE DRAWINGS

In order to facilitate a more detailed understanding of the nature of the inventions disclosed herein, exemplary embodiments of systems, methods and apparatuses for rehabilitation of auditory system disorders will now be described in detail, by way of example only, with reference to the accompanying drawings in which:

FIG. 1 is a graphical representation of the long-term spectra of both a music recording and a typical prior art tinnitus masker;

FIG. 2 is a diagram illustrating the contour changes associated with auditory system disorders in accordance with certain exemplary embodiments disclosed herein;

FIG. 3 is a graphical representation of an exemplary patient's hearing thresholds and their required equalization curve calculated in accordance with certain exemplary embodiments disclosed herein;

FIG. 4 is a schematic diagram graphically illustrating intermittent tinnitus relief with music in accordance with certain exemplary embodiments disclosed herein;

FIG. 5 is a graphical representation of an exemplary patient's hearing thresholds and their required equalization curves calculated in accordance with certain exemplary embodiments disclosed herein;

FIG. 6 is an exemplary illustration of tinnitus pathogenesis and mechanism of action in accordance with certain exemplary embodiments disclosed herein;

FIG. 7 is a schematic diagram of an exemplary tinnitus rehabilitation method in accordance with certain exemplary embodiments disclosed herein;

FIG. 8 is a schematic block diagram of a possible embodiment of an auditory system disorder rehabilitation device in accordance with certain exemplary embodiments disclosed herein;

FIGS. 9A-E and 10A-D are flowcharts illustrating an exemplary method of providing an auditory system disorder rehabilitation sound recording in accordance with certain exemplary embodiments disclosed herein;

FIG. 11 is a schematic diagram of an embodiment of an auditory system disorder rehabilitation device in accordance with certain exemplary embodiments disclosed herein;

FIG. 12 is a schematic diagram of an exemplary tinnitus rehabilitation method in accordance with certain exemplary embodiments disclosed herein;

FIG. 13 is a schematic diagram of an exemplary tinnitus rehabilitation method in accordance with certain exemplary embodiments disclosed herein;

FIG. 14 is a functional schematic of a digital playback device in accordance with certain exemplary embodiments disclosed herein;

FIG. 15 a schematic diagram of an exemplary tinnitus rehabilitation system in accordance with certain exemplary embodiments disclosed herein; and

FIG. 16 is a functional schematic of a digital audio distribution system in accordance with certain exemplary embodiments disclosed herein.

### DETAILED DESCRIPTION

Certain embodiments provide a more effective rehabilitation technique for people suffering from auditory system disorders based on more contemporary understandings of the neural processes underlying the auditory system disorders.

Such systems, methods and apparatus may, in certain embodiments, provide relief from the perception of the auditory system disorder (for example, tinnitus). The relief may be provided by masking (e.g., covering up) the perception of the auditory system disorder. However, relief may also be achieved by certain embodiments without complete masking. In certain embodiments, the relief from the perception of the auditory system disorder (for example, desensitization and/or habituation of the auditory system disorder) may be achieved by modification of the audio signal which may provide the person with, for example, relaxation, stimulation of the auditory system, partial or intermittent masking, interaction with the perception of the auditory system disorder, inhibition or partial or intermittent inhibition, distraction, or some combination thereof. Additionally, in certain embodiments, users may receive relief from the perception of the auditory system disorder, arising from stimulation of auditory pathways in regions of hearing loss and/or interaction with their tinnitus perception to a sufficient degree that reduces their attentional focus on the perception of the auditory system disorder.

According to certain embodiments, there are provided methods for providing relief to a person suffering from the effects of an auditory system disorder (for example, tinnitus and/or conditions of reduced tolerance to loud sounds), comprising: providing at least one audio signal spectrally modified in accordance with the person's profile designed to modify the intensity of the audio signal at certain frequencies whereby, in use, when the at least one spectrally modified audio signal is heard by the person it provides some amount of relief of the auditory system disorder. The person's profile may be predetermined.

According to certain embodiments, there are provided methods for providing relief to a person suffering from the effects of an auditory system disorder (for example, tinnitus and/or conditions of reduced tolerance to loud sounds), comprising: means for providing at least one audio signal spectrally modified in accordance with the person's predetermined profile designed to modify the intensity of the audio signal at certain frequencies whereby, in use, when the at least one spectrally modified audio signal is heard by the person it provides some amount of relief of the auditory system disorder.

In certain embodiments, the person's predetermined profile may be unique to the individual person or may be selected from a set of preselected profiles. For example, as will be discussed in more detail below, the profile may be a person's audiogram or audiogram approximation or may be a generic or standard profile that is selected (or created) by the person (or someone else, including, but not limited to, a clinician, healthcare professional, or computer) as being an acceptable match for the person's auditory system disorder. The resulting profile can be used to spectrally modify the audio signal (for example, but not limited to, by using a predetermined algorithm).

In certain embodiments, at least one, two, three, or four profiles may be generated, provided and used at various stages of the treatment. In certain embodiments, the profile and/or profiles provided may be further adjusted by the user or some other third party for use at different stages of the treatment. In certain embodiments, the profile and/or profiles provided may be adjusted (or remain the same) and used with different audio sources at different stages of treatment. For example: white noise, TV, music; music, TV, white noise; music, TV; TV, music; or other combinations of audio sources depending on the particular treatment and/or patient. For example, the profile may used with a music signal at an initial stage of treatment to substantially mask the auditory system disorder and provide initially relief to the patient and this could be followed by using the same or a different profile with an television audio signal in a later stage of treatment to partially mask the auditory system disorder and to provide effective long term habituation. It is of course possible to switch back and forth and combine different profiles and different audio sources throughout the different stages of the treatment process. These profiles may be unique to the individual person, selected from a set of preselected profiles, a profile based on a person's audiogram, a profile based on an audiogram approximation, a generic or standard profile that is selected (or created) by the person (or someone else, including, but not limited to, a clinician, healthcare professional, or computer) as being an acceptable match for the person's auditory system disorder, or combinations thereof. For example, it may useful in certain treatments to provide a particular profile that provides an effective early sense of relief from the auditory disorder such the person being treated starts to feel some control over the disorder and/or starts to relax. This may be followed up with a second profile that is effective for habituation and long term treatment to occur. In certain embodiments, this may be followed up with the third profile that is effective in the later stages of the treatment. For example, it may be desirable in certain situations to start with a profile that has an acoustic signal that is relaxing enough but rich and distracting enough to reduce perception. This may be followed up with a profile that has effective dynamic intensity over time to be effective at both relaxation and habituation.

Certain methods may comprise: transmitting data representing an audiogram or audiogram approximation of the person suffering from the auditory system disorder; processing the audiogram or audiogram approximation data (for example, at a remote location, on a personal computer, or in the device) and producing required equalization response data based on the audiogram or audiogram approximation data using the predetermined profile; receiving the required equalization response data; and, combining the required equalization response data with audio data representing the audio signal to produce the spectrally modified audio signal.

Certain methods may comprise: means for transmitting data representing an audiogram or audiogram approximation of the person suffering from the auditory system disorder; means for processing the audiogram or audiogram approximation data (for example, at a remote location, on a personal computer, or in the device) and means for producing required equalization response data based on the audiogram or audiogram approximation data using the predetermined profile; means for receiving the required equalization response data; and, combining the required equalization response data with audio data representing the audio signal to produce the spectrally modified audio signal.

According to certain embodiments there is provided a method of using a computer to provide access to a predetermined algorithm used in auditory system disorder rehabilitation, for providing relief to a person suffering from the disturbing effects of an auditory system disorder, comprising: receiving on-line, from a user, data representing an audiogram or an audiogram approximation of the person suffering from tinnitus; processing the audiogram or audiogram approximation data using the predetermined algorithm to produce required equalization response data based on the audiogram or audiogram approximation data; and, transmitting the required equalization response data to the user.

According to certain embodiments there is provided a method of using a computer to provide access to a predetermined algorithm used in auditory system disorder rehabilitation, for providing relief to a person suffering from the disturbing effects of an auditory system disorder, comprising: means for receiving on-line, from a user, data representing an audiogram or an audiogram approximation of the person suffering from tinnitus; means for processing the audiogram or audiogram approximation data using the predetermined algorithm to produce required equalization response data based on the audiogram or audiogram approximation data; and, means for transmitting the required equalization response data to the user.

According to certain embodiments, there is provided an auditory system disorder rehabilitation sound recording for providing relief to a person suffering from the disturbing effects of tinnitus, comprising: an audio signal spectrally modified in accordance with an algorithm designed to modify the intensity of the audio signal at selected frequencies whereby, in use, when the sound recording is heard by the person it provides some amount of relief of the auditory system disorder. In certain embodiments, there is provided an auditory system disorder rehabilitation sound recording for providing relief to a person suffering from the effects of tinnitus, comprising: at least one audio signal spectrally modified in accordance with a spectral response related to the patient's audiogram using an algorithm and/or by using a graphic equalizer or by selecting a spectral response from library of responses designed to modify the intensity of the audio signal at least one, two, three, four, five six, or seven selected frequencies whereby, in use, when the sound recording is heard by the person it provides some amount of relief of the auditory system disorder. In certain embodiments, there is provided an auditory system disorder rehabilitation sound for providing relief to a person suffering from the effects of tinnitus, comprising: at least one audio signal spectrally modified in accordance with an algorithm and/or other means designed to modify the intensity of the audio signal in about 100%, about 90%, about 80%, about 70%, about 60%, about 50%, about 40%, about 30%, about 20%, about 10%, about 5%, or about 1% of the frequencies provided whereby, in use, when the sound is heard by the person it provides some amount of relief of the auditory system disorder. In certain embodiments, there is provided an auditory system disorder rehabilitation sound for providing relief to a person suffering from the effects of tinnitus, comprising: at least one audio signal spectrally modified in accordance with an algorithm and/or other means designed to modify the intensity of the audio signal in 100% to 10%, 60 to 5%, 90% to 40%, 80% to 10%, 70% to 30%, 60% to 20%, 50% to 5%, 40% to 1%, 30% to 4%, 20% to 5%, 10% to 2%, 5% to 1%, of the frequencies provided whereby, in use, when the sound is heard by the person it provides some amount of relief of the auditory system disorder.

In certain embodiments, the predetermined algorithm may provide intermittent relief of the tinnitus wherein, at a comfortable listening level, during peaks of the audio signal the tinnitus is substantially completely obscured, whereas during troughs the perception of the tinnitus occasionally emerges. In certain embodiments, the predetermined algorithm may provide intermittent relief of the tinnitus wherein, at a comfortable listening level, during a substantial number of the peaks of the audio signal the tinnitus is at least partially obscured. In certain embodiments, the predetermined algorithm may provide an effective amount of relief of the tinnitus. In certain embodiments, the predetermined algorithm may provide an effective amount of relief of the tinnitus wherein, during troughs the perception of the tinnitus may be perceived. In certain embodiments, the predetermined algorithm may provide an effective amount of relief of the tinnitus wherein, at desired listening levels, during a number of the peaks of the audio signal the tinnitus is partially, substantially, or completely obscured, whereas during troughs the perception of the tinnitus emerges, partially emerges, or substantially emerges. In practice it has often been found that such intermittent relief can provide an immediate sense of relief, control and relaxation for the person, whilst enabling sufficient perception of the tinnitus for habituation and long term treatment to occur.

In certain embodiments, the predetermined algorithm may be designed to modify the intensity of the audio signal across substantially the full spectral range of the audio signal. The audio signal may be a highly dynamic signal in which the spectral content and intensity constantly or substantially varies over time. The audio signal may be, but is not limited to, a music signal. The audio signal may be the audio associated with a television broadcast or other audio visual content such as movies, sport programs, news programs, other acceptable audio visual signals, or combinations thereof. Other types of signals are contemplated, including speech, tones, or noise may also be employed. The audio signals may be combined in a number of ways to produce an acceptable listening experience for the user. In addition, the audio and visual signals may be combined in a number of ways to produce an acceptable experience for the user. In certain embodiments, combining audio with visual may provide an effective treatment for the patient. The audio signals may be further modified so as to reduce the dynamic range and provide better opportunity for the patient to listen at a volume level that provides more effective stimulation in the region of hearing loss. The patient may find that the visual stimulation is effective at relaxing and/or distracting the patient such that the audio stimulation is more effective at reducing the initial perception of the auditory disorder and/or the visual stimulation permits more effective habituation for long term treatment. In some aspects the treatment may involve using audio treatment at certain stages and audio/visual treatment at other stages of the treatment.

In certain embodiments, the predetermined algorithm may be tailored, partially tailored, or substantially tailored to the audiometric configuration of the person. The predetermined algorithm may be tailored, partially tailored, or substantially tailored to the hearing loss characteristic of the person. The spectral qualities of the audio signal may be modified by the predetermined algorithm so as to provide a relatively equal, or at least approximately perceptually balanced, sensation level across a major portion of the audio spectrum in both ears, or at least a sufficient portion to create a perception of distributed sound field, rather than a perception of an isolated sound source in order to facilitate stimulation of the auditory system within a pleasant and comfortable listening experience. The spectral qualities of the audio signal may be modified by the predetermined algorithm so as to account for reduced sound tolerance and/or loudness recruitment. For example, the predetermined algorithm may adjust for somewhat less than the full degree of hearing loss, or may include compression or other means to ameliorate peaks of intensity of other unpleasant features of the acoustic stimulus.

In certain embodiments, an audiologist may take a patient's full audiogram and use that data to create a profile which is used to produce the spectrally modified music or other audio signal. In certain embodiments, an audiologist may take a patient's full and/or partial audiogram and use that data to create a profile which is used to produce the spectrally modified audio signal.

Alternatively, a self and/or partially self administered audiogram and/or audiogram approximation may be used to create a predetermined profile which is used to produce the spectrally modified music or other audio signal. For example, the patient may be provided with tones or bands of noise and be instructed to turn up volume until the tone or band of noise is audible. Alternatively, the tones may be provided with random amplitudes and frequencies and the patient may be instructed to indicate whether it is audible or not. In certain embodiments, the patient may be provided with graphic equalizer functionality to select an appropriate equalization based on patient preference.

In certain embodiments, a set of profiles may be used and one such profile selected to create a predetermined profile which is used to produce the spectrally modified music signal (or the selected profile may be used to directly create a modified audio signal). In certain aspects, the profiles may be selected from or comprise a standard or generic set of profiles. For example, a patient may be instructed to select a profile from a number of profiles either by way of trial and error or by way of a defined interface or some logical process (for example, a hierarchical tree structure or decision tree) for determining the best profile. In certain embodiments, the system may use one of a number of predetermined profiles based on the audiogram approximation. In certain embodiments, the system may include a single adjustable profile that can be adjusted to patient preference.

According to certain embodiments, there is provided an auditory system disorder rehabilitation device for providing relief to a person suffering from the disturbing effects of the auditory system disorder comprising: signal filtering means adapted to spectrally modify an audio signal in accordance with a predetermined profile designed to modify the intensity of the audio signal at selected frequencies whereby, in use, when the spectrally modified audio signal is heard by the person it provides some amount of relief of the auditory system disorder.

According to certain embodiments, there is provided an auditory system disorder rehabilitation device for providing relief to a person suffering from the disturbing effects of the auditory system disorder comprising: means for signal filtering adapted to spectrally modify an audio signal in accordance with a predetermined profile designed to modify the intensity of the audio signal at selected frequencies whereby, in use, when the spectrally modified audio signal is heard by the person it provides some amount of relief of the auditory system disorder.

In certain embodiments, the signal filtering means may be a programmable signal filtering means whereby, in use, the device can be programmed with a predetermined algorithm adapted to the particular needs of the individual suffering from tinnitus.

In certain embodiments the predetermined algorithm may be of the form:

REQ=M(SPL+ELC(0.25,0.5,1,2,3,4,6,8,10,12 kHz)−Baseline)

Where:

REQ=Required equalization response of the Tinnitus Retraining Protocol (TRP)

Baseline=0.5 (A−B)+B

A=mean dB SPL at the two adjacent greatest hearing loss frequencies in the greatest hearing loss ear

B=mean dB SPL at the two adjacent least hearing loss frequencies in the least hearing loss ear

SPL=hearing thresholds (in dB HL) converted to dB SPL

ELC=transfer values for 40 Phon Equal Loudness Contours

M=gain multiplier=0.3 to 0.95, and

Preferably M=0.4

In certain embodiments the gain multiplier may be between 0.3 to 0.5, 0.0 to 1.0, 0.2 to 0.6, 0.1 to 0.7, 0.2 to 0.8, 0.4 to 0.9, about 0.25, about 0.30, about 0.35, about 0.40, about 0.45, about 0.50, about 0.55, or about 0.60.

In certain embodiments, the mathematical algorithm by which the individual prescription of the audio signal is calculated may differ from the above algorithm.

The device may be employed in conjunction with a personal music player (PMP), television or other audio visual device and have an input adapted to connect to the audio output of the PMP, television or other audio visual device. The device may have a standard headphone jack to which a standard PMP headphone can be connected.

Alternately, a transmitter may be used to transmit a signal to a wireless type of receiver that may be placed in the device, in the ear canal, concha area, behind the ear, or some other area relatively close to the ear.

In certain embodiments, there is provided a digital playback device that includes at least one processor for receiving the audio signal, spectrally modifying the audio signal to compensate for an auditory system disorder, and outputting a spectrally modified audio signal to a user.

In certain embodiments, there is provided a digital playback device that includes means for receiving the audio signal, means for spectrally modifying the audio signal to compensate for an auditory system disorder, and means for outputting a spectrally modified audio signal to a user.

In certain embodiments, there is provided a method for distributing an audio signal that includes modifying the audio signal with one of a number of preselected spectral modification signals and/or algorithms to create a spectrally modified audio signal and then, after modification, providing the spectrally modified audio signal to the user.

In certain embodiments, there is provided a method for distributing the audio signal that includes obtaining user information at a kiosk to determine which of a number of spectral modification signals and/or algorithms to use to create the spectrally modified audio signal before distributing the audio signal to the user.

Exemplary embodiments of systems, methods and apparatuses for rehabilitation of auditory system disorders by providing a stimulus to the auditory system of an individual experiencing an auditory disorder are described herein.

Tinnitus “masking” may be broadly defined as the obscuring, or partial obscuring, of tinnitus perception with an external sound. An accepted audiometric measure of the effectiveness of tinnitus maskers is the intensity of sound required to just mask an individual's tinnitus. This measure is known as the Minimum Masking Level (“MML”). One criterion for successful masking is that the acceptability of a masking stimulus be inversely proportional to its MML, and that the stimulus be a sufficiently pleasant substitute for the tinnitus. During clinical practice, the inventors have observed that several tinnitus sufferers have reported attempting to use music to find relief from their tinnitus, but often found that the volume required to mask their tinnitus was unacceptably high. It was also observed that most of these persons tended to have a steeply sloping hearing loss characteristic, and a tinnitus pitch which closely corresponded with the edge of the maximal hearing loss frequencies. Therefore, one of the reasons why previous attempts at using music have not always been successful may be the high co-morbidity of high frequency hearing loss with tinnitus.

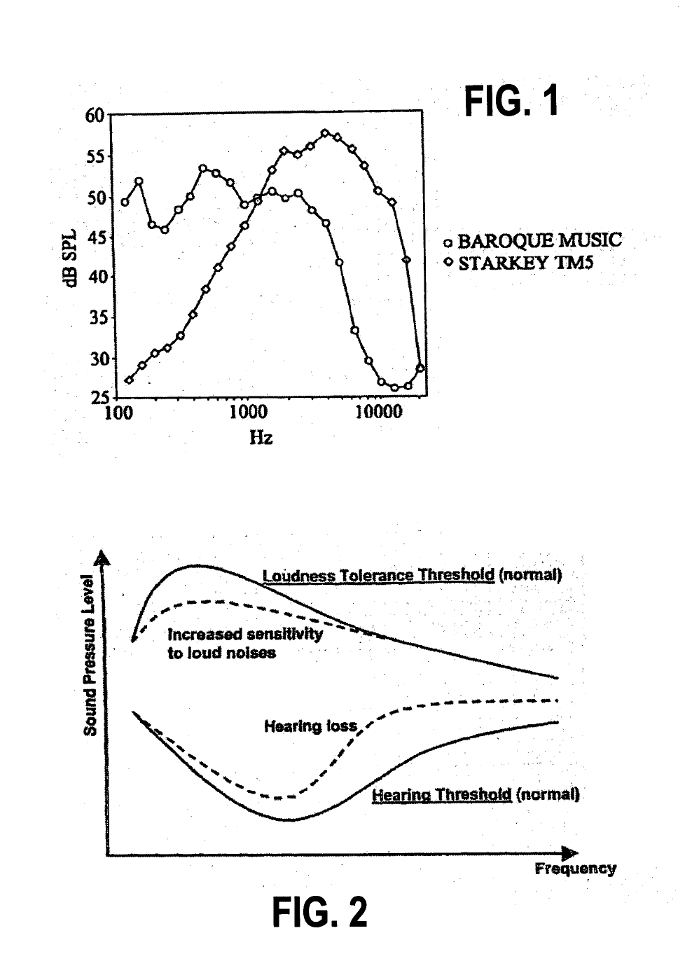

Typically, the presence of a sloping high frequency hearing loss would mean that at a relaxing sound volume level, only the low pitch components of the music are heard, and therefore the perception of full musicality and high frequency energy available for masking is inhibited. The long term spectra of both a music recording and a typical prior art tinnitus masker (e.g., a Starkey TM5) are illustrated in FIG. 1. In FIG. 1, a sound level analyzer was used to average the response of each of the two recordings over a 64 second period. The spectra were then matched at 1 kHz to enable a comparison of the frequency composition of the two spectra, irrespective of overall sound pressure levels. As can be seen from FIG. 1, if the masker is assumed to be the optimal frequency response for hearing impaired listeners, then the unfiltered music has insufficient high frequency energy and excessive low frequency response. After over a decade of research into the effect of various acoustic stimuli on auditory system disorders, an understanding of the brain changes which contribute to auditory system disorders (including but not limited to, for example, tinnitus, hyperacusis, and hearing loss) and recognition that the auditory system disorder may be different for everyone, it has been determined that an important aspect of providing tinnitus relief is providing stimulation to auditory pathways deprived of stimulation as a result of hearing loss. For example, as illustrated in FIG. 2, an individual suffering from hearing loss may have an above normal hearing threshold. In addition, the individual may also have an increased sensitivity to loud noises (e.g., hyperacusis, etc) which results in a lower than normal threshold for louder sounds at certain frequencies. The upper threshold (the loudness tolerance threshold), as seen in FIG. 2, may be most prominent at frequencies that are different from the frequencies where hearing loss occurs. Accordingly, exemplary embodiments disclosed herein are for a tinnitus protocol which modifies the frequency response characteristics of an audio signal with a view to overcoming some of the shortcomings of traditional tinnitus maskers. In certain embodiments, these shortcomings may include, for example, a lack of higher frequency signals, sounds that are not pleasant to listen to, and sounds that need to be listened to at an uncomfortably loud level, insufficient energy at frequencies where the patient has hearing loss, or excessive energy at frequencies where the patient has reduced tolerance of loud sounds.

Although the following description will be made primarily with reference to modifying the frequency response characteristics of music, it is to be understood that a tinnitus protocol in accordance with the disclosed inventions may also be applied to other types of audio signals suitable for relieving tinnitus, or for providing auditory stimulation for tinnitus and hyperacusis therapy. For example, noise with a large bandwidth (e.g., broad band noise, white noise or substantially white noise) or pure tones may be used as well and in certain embodiments, for example, white noise may be combined with for example, music or other sounds (e.g., speech) to create a combined audio signal.

Additionally, the embodiments described herein give particular emphasis to the use of conventional, insert or wireless headphone systems or insert type headphones in conjunction with a suitable personal sound reproduction system such as a high fidelity personal music player (PMP) for audio cassette, CD, MP3, or WMA recordings. The cost of a high fidelity PMP may be around one-tenth the cost of, for example, conventional binaural maskers. However, it is to be understood that the tinnitus protocol according to the invention may also be applicable to conventional hearing aid-style devices, maskers and/or combinations thereof. The technique can also be applicable to the setting of additional user programs in hearing aids, or the modified signal may be transmitted to the tinnitus sufferer through their hearing aids' telecoil or induction coil facility. Additionally, conventional loud speakers may be used in certain embodiments. However, in certain embodiments, use of conventional loud speakers may not be desirable to use in certain systems for reasons that will be apparent to persons of ordinary skill in the art based on this disclosure.

In addition, PMPs generally possess small headphones with long-throw transducers that enable far superior fidelity compared to most free field loudspeaker. Headphones are generally more effective than loud speakers because they circumvent the extensive attenuation of high frequency sounds that occurs through a free field.

While developing an exemplary protocol, the required extended upper frequency stimulus presented challenges for the conversion of audiogram results to the required real ear response, given that there were no internationally agreed-upon standards for the conversion between dB HL to dB SPL for 10 and 12 kHz pure tone and narrow band noise stimuli. Although there were no agreed upon standards at the time these conversions were initially required, today, the conversions may be provided for by, for example the ISO-TR/389-5 standard and in certain embodiments, this standard may be used for the calibration process.

The manufacturer's calibration specifications for a Madsen OB 822 audiometer were used to extrapolate the required values for use with a telephonics TDH 39 headphones and MX 41/AR cushions. The audiometer was professionally calibrated accordingly. The values for 10 kHz were 50 dB HL=59.5 dB SPL and at 12 kHz, 50 dB HL=61 dB SPL. All ISO hearing level frequencies below 10 kHz were calibrated as per the relevant Australian standards (AS 1591.2—1987). Table 1 lists the transfer/calibration values in inverted format used for converting dB HL to dB SPL.

TABLE 1

| | Frequency | | | | | | | | | | |
|---|---|---|---|---|---|---|---|---|---|---|---|
| kHz | 0.25 | 0.5 | 1 | 1.5 | 2 | 3 | 3 | 6 | 8 | 10 | 12 |
| dB | 25.5 | 11.5 | 7.0 | 6.5 | 9.0 | 10.5 | 10.5 | 16.5 | 12.0 | 9.5 | 11.0 |

A further feature of the this exemplary tinnitus protocol (TP1) developed by the inventors, was an adaptation of the so called “half gain rule”, whereby amplification for hearing loss compensates for only around one half of the hearing deficit. This rule underlies most current hearing aid prescriptive practices. The TP1 attempted to maximize the acoustic energy centered around the pitch of the individual's tinnitus, and to “balance” the headphone output to correct for any asymmetrical hearing loss. A further goal was to enable the balanced perception of the stimulus throughout the person's head, rather than at the ear level like traditional uncorrelated devices.

PMPs generally have a volume control range that exceeds what is available in hearing aids, and so the TP1 did not need to specify absolute gain figures. However, PMPs generally do not possess a left/right balance control, and this was expected to reduce their acceptability in cases of asymmetrical hearing loss and its associated loudness recruitment. As the TP1 formulae aimed to minimize the perceptual loudness of the music or noise or other acoustic stimulus required to provide some amount of relief of an individual's perception of tinnitus, it thus only needed to specify the relative frequency response characteristics for each ear when presented in those reproduction systems that do not provide individual control of each stereo channel. Maintaining discrete signals for each ear, and controlling the temporal correlation between those signals is advantageous because it allows any asymmetry in the auditory system disorders (e.g., levels of hearing loss) exhibited by the two ears to be accounted for. In this way, the degree of customization for the auditory system disorder is enhanced. This allows maximum stimulation in frequencies of hearing loss while at the same time ensuring a pleasantly low listening volume, thereby enhancing the listening experience for the user. By allowing a stereo, and hence a spatially distributed and more engrossing listening experience, user acceptability is further enhanced. A further advantage is that the integrative pathways of the auditory system are stimulated in this way.

The procedure for applying the TP1 was thus as follows:

(i) The individual's pure tone hearing level thresholds at each frequency were converted to dB SPL by the addition of the transfer values in Table 1.

(ii) The tinnitus pitch match frequency in the most severely affected ear was chosen for the maximal point of the base line calculation. The two adjacent best hearing thresholds of the lesser hearing loss ear was always chosen as the minimum point of the calculation. When a reliable pitch match was not found using pure tones, it was substituted with the mean of the two adjacent best hearing frequencies. Thus, the base line constituted a mid line value between the two greatest audiometric extremities.

(iii) The final equalization values were then derived by subtracting the base line from the hearing threshold (expressed in dB SPL) for each frequency and each ear. Thus the algorithm for patients whose tinnitus pitch could not be reliably determined was:

Baseline=0.5(A−B)+B

Required Equalization, REQ=0.5{SPL(0.25,0.5,1,2,3,4,6,8,10,12 kHz)−Baseline}

The algorithm for non-tonal tinnitus was:

Baseline=0.5(C−B)+B

REQ=0.5{SPL(0.25,0.5,1,2,3,4,6,8,10,12 kHz)−Baseline}

Wherein,

A=hearing threshold (dB SPL) at frequency of tinnitus pitch match.

B=mean dB SPL at the 2 adjacent least hearing loss frequencies.

C=mean dB SPL at the 2 adjacent greatest hearing loss frequencies.

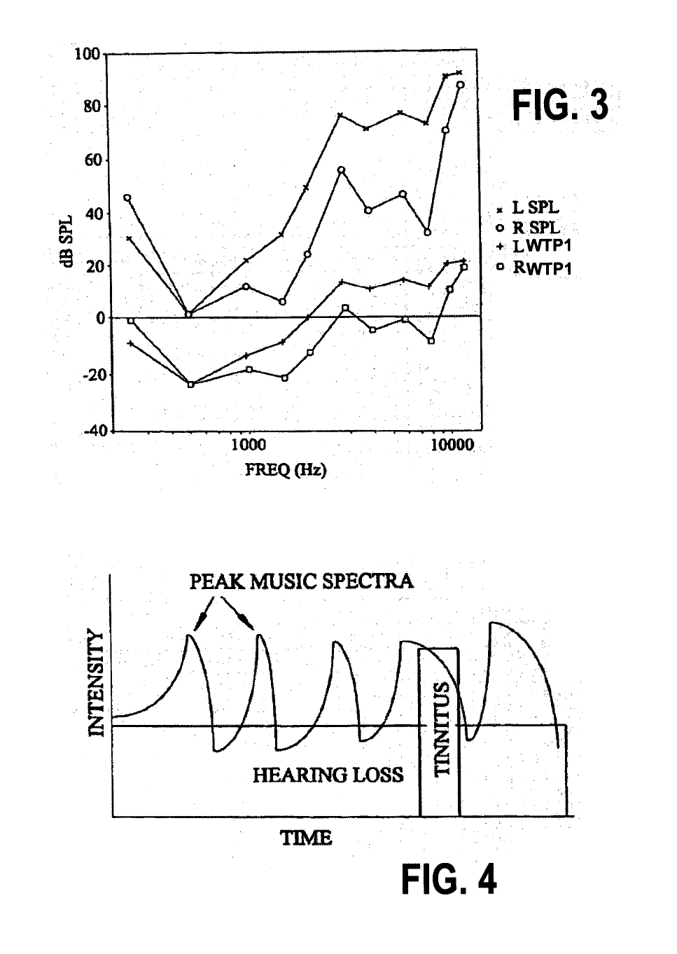

FIG. 3 is a graphical representation of an exemplary relationship between a typical individual's hearing levels, tinnitus and the TP1 “equalization curves”. As seen in FIG. 3, this individual has a steeply sloping high frequency bilateral hearing loss and tinnitus at 10,000 Hz, both greater on the left side. Consequently, the required equalization curves revolve around the equalizer's baseline, achieving a partial correction for hearing loss by boosting the amount of high frequency gain and also correspondingly attenuating the low frequencies. As the hearing loss and tinnitus is worse on the left, that ear receives correspondingly greater amplification. Because of the abnormal growth of loudness perception which usually accompanies sensorineural hearing loss, (recruitment, and/or the presence of hyperacusis), complete correction for hearing levels is not provided, as this may exceed the individual's loudness discomfort levels.

A tinnitus rehabilitation sound recording was then produced on an audio cassette tape for use in the individual's PMP. A stereo frequency equalizer (Genexxa 31-9082) was used in this procedure, which includes ten adjustable frequency bands per channel, with centre frequencies at 0.031, 0.062, 0.125, 0.25, 0.5, 1, 2, 4, 8, 16 kHz. Each control had a range of + or −12 dB SPL. The equalizer featured an “EQ record” facility, so that the audio signal could be passed through the equalizer circuit before being recorded. The equalizer's controls for each of the ten frequency bands was set to the calculated values for the left ear in the left channel of the equalizer, and the right ear values set in the right channel, in accordance with the particular individual's equalization values as calculated by the TP1 algorithm. The stereo output from a broadcast quality cassette recorder (or, in certain embodiments, any type of audio visual storage medium) was connected to the stereographic equalizer, which then had its output routed to another high fidelity cassette deck (or, in certain embodiments, any type of audio visual storage medium) for recording onto high fidelity audio cassette tape. Dual leads and stereo RCA connecters were used to preserve L/R channel separation in light of the advantages of a stereo signal with separately customized signals as described herein.

Modified sound recordings of both music and white noise were made for use in clinical trials with 30 participants. Each participant was counseled as to the rationale behind the therapy and the possible benefits of using the tinnitus rehabilitation sound recording. Each participant was issued a new PMP with standard insert headphones (Sony MDR E552) that fit into the concha and thus do not require a headband. Sound level-peak analysis measures were then performed. With their custom-made tape playing in the PMP, they were asked to slowly turn up the volume until they could just no longer perceive their own tinnitus. This level was marked on the volume control wheel. Each participant was told to notify the audiologist if they subsequently needed to turn up the volume further than the marked position. They were encouraged to experiment downwards with the volume control over the course of each session, as they might find that they require progressively less volume to provide relief if, for example, residual masking occurred.

One group of participants was given a noise tape whereas the other group was given a music tape. While both treatment groups had similar levels of pre-therapy distress associated with their tinnitus, the music group displayed a greater improvement by mid-therapy and these gains were maintained at a two-year post-therapy follow up. The noise group also displayed some improvement, but less dramatic then the music group. 96% of the participants found their music or noise tapes to be an effective device for providing relief.

In some cases, the TP1 appeared to present an unbalanced perception of loudness where the individual possessed a substantial inter-aural asymmetry. The real-ear perception of loudness may have deviated from the prescribed response due to perception of loudness differences at various points across the frequency range. It was also thought that the half gain rule for hearing aids might be best suited for the moderate hearing loss population, and that a mild hearing loss might only require one-third gain. Furthermore, it is possible that the recruitment of loudness phenomena might be greater in tinnitus patients than non-tinnitus patients, particularly given its high co-morbidity with hyperacusis and phonophobia (the fear of external sounds). These factors suggested that the TP1 might be over-compensating for hearing loss, and that further modifications were required to optimize the procedure.

An objective of the TP1 algorithm was to produce an acceptable substitute for the tinnitus at the lowest possible MML and to accommodate for any interaural symmetries. However, it was subsequently realized that an improved algorithm would be more robust if the prescription of the required equalization response was performed on the basis of maximum and minimum hearing levels, and thereby attempt to provide relatively equal sensation levels at all frequencies. Data from the TP1 study indicated that 44.4% of the music group, and 28.6% of the noise group participants preferred to set the volume of their audio tapes at a level which provided relief by only partially masking their tinnitus perception. This occurred despite being instructed that the optimal setting was to totally mask their tinnitus. The differences in masking level preferences between the two types of stimuli also suggests that music was more acceptable than noise when used at volume levels where the tinnitus could still be partly perceived. Whilst certain embodiments may totally mask tinnitus, other embodiments may partially mask. In fact, the present inventors have developed an improved tinnitus protocol based on providing relief via intermittent masking. Since music is a dynamic signal, it appears possible that the intensity of music which partially masks might actually constitute a form of intermittent masking. A schematic representation of intermittent tinnitus masking using a music signal is illustrated in FIG. 4.

It is believed that the providing relief of the perception of tinnitus with a relaxing stimulus (such as music) may be effective, by virtue of the distraction provided, on a psychological, as well as on an acoustic or neural level. In theory, it is feasible that providing relief with music might constitute a form of systematic desensitization. Whilst in a relaxed state, the listener might be alternatively perceiving, then not perceiving the tinnitus, according to the fluctuations in the peak levels of the music. The predictability of the music may mean that the tinnitus might not even be consciously perceived during the “troughs” of the music. Additionally, the tinnitus might “reappear” from the music often enough for habituation to occur. But the ongoing dynamic nature of the music signal prevents this limited exposure from being disturbing, and this may reduce any limbic system enhancement and/or conscious attentional focus on the tinnitus perception. Thus, the proposed relief (e.g., resulting from the intermittent-masking-with-relaxation-music technique) may promote a synergistic effect through its additional mechanisms of facilitating a sense of control, a reduction in general anxiety levels, and/or a form of auto-hypnosis leading to a reduction of fear about the tinnitus itself. Therefore, another exemplary algorithm based on a tinnitus retraining protocol (TRP) was developed that was designed to produce intermittent masking of the tinnitus.

In practice, the TP1 algorithm's use of the so-called half-gain rule appeared to over-compensate for hearing loss as noted above, sometimes making the recording seem unbalanced or “tinny”. Conversely, there were several factors that suggested that one-third gain might not provide sufficient equalization. The long term music spectrum has considerably less high frequency energy than what is typically available from conventional tinnitus maskers, and yet the greatest hearing loss is typically concentrated in this region (see FIG. 1). Therefore, any substantial reduction of gain could prevent achieving adequate high frequency equalization to overcome the limitations in the music spectra and the effects of hearing loss. Therefore, because the so called half gain rule was sometimes excessive, but one third gain may be insufficient in certain situations for the purposes of modifying music for long term tinnitus retraining, a medium was selected by the incorporation of a 0.4 gain multiplier, (M). In certain embodiments the gain multiplier may be between 0.3 to 0.5, 0.0 to 1.0, 0.2 to 0.6, 0.1 to 0.7, 0.2 to 0.8, 0.4 to 0.9, about 0.25, about 0.30, about 0.35, about 0.40, about 0.45, about 0.50, about 0.55, or about 0.60.

To further facilitate the provision of equal sensation levels of music across the full spectral range of the music signal, the TRP algorithm adopted the ISO Equal Loudness Contours (ELC). The ELC transfer values correct for any differences in loudness perception depending on the discreet frequencies (International Standards Association, 1961). The 40 phon contour curve was selected because the earlier study found that the mean participant's customized music recordings, under total masking conditions, displayed a RMS of 45.7 dB SPL. Thus, with 8 dB representing an approximate doubling of perceived loudness, 37.7 dB was extrapolated to be the midpoint between the threshold and total masking, and thus representative of the intensity around which intermittent masking would occur with those with a mild to moderate sloping hearing loss. The 40 phon contour was thus utilized because it was the closest to this mid point, and choice of the lower value curve also helped compensate for loudness recruitment.

One standard audiometric procedure is to obtain hearing thresholds using TDH 39 headphones, and the results are expressed in dB HL (Hearing Level). However, one convention for specifying hearing aid characteristics is to utilize dB SPL (Sound Pressure Level) values. Consequently the hearing thresholds (dB HL) obtained in the 6 cm³ headphones need to be converted into dB SPL by the addition of the transfer values in Table 1.

These transfer values were then summated with the 40 Phon contour values. The resulting transfer/calibration values are displayed in Table 2.

TABLE 2

| | Frequency | | | | | | | | | | | |
|---|---|---|---|---|---|---|---|---|---|---|---|---|
| kHz | .25 | .5 | .75 | 1 | 1.5 | 2 | 3 | 4 | 6 | 8 | 10 | 12 |
| Corrections | 23.5 | 7.5 | 5.5 | 7 | 6.5 | 7 | 5.5 | 2.5 | 16.5 | 21 | 16.5 | 13 |

The tinnitus retraining protocol (TRP) algorithm is a modification of the TP1 algorithm given above, and is as follows:

REQ=0.4{ELC+SPL(0.25,0.5,1,2,3,4,6,8,10,12 kHz)−Baseline}

Where:

Baseline=0.5 (A−B)+B

A=mean dB SPL at the two adjacent greatest hearing loss frequencies in the greatest hearing loss ear.

B=Mean dB SPL at the two adjacent least hearing loss frequencies in the greatest hearing loss ear.

SPL=hearing thresholds (in dB HL), converted to dB SPL.

ELC=Transfer values for 40 Phon Equal Loudness Contours.

Alternatively, the patient's hearing thresholds may be obtained using ⅓ octave narrow band noises, and the gain multiplier (M) becomes 0.7 (or between the range of 0.5 to 0.95, 0.3 to 0.5, 0.4 to 1.0, 0.2 to 0.6, 0.4 to 0.7, 0.4 to 0.8, 0.4 to 0.9, about 0.40, about 0.45, about 0.50, about 0.55, about 0.60, about 0.75, about 0.9, or about 1). In certain embodiments the gain multiplier may be between about 0.3 to 0.5, 0.0 to 1.0, 0.2 to 0.6, 0.1 to 0.7, 0.2 to 0.8, 0.4 to 0.9, about 0.25, about 0.30, about 0.35, about 0.40, about 0.45, about 0.50, about 0.55, about 0.60, about 0.7, about 0.8, about 0.9, or about 1.0.

The procedure for applying the TRP was as follows:

(i) The person's audiogram was perused to ascertain the two adjacent greatest hearing loss frequencies in the greatest hearing loss ear (A), and also the two adjacent least hearing loss frequencies in the least hearing loss ear (B).

(ii) These four dB HL values were then converted to dB SPL by the addition of the transfer values in Table 1.

(iii) The dB SPL mean of the two adjacent greatest hearing loss frequencies in the greatest hearing loss ear (A) was then calculated in dB SPL, and the procedure repeated for the two adjacent least hearing loss frequencies in the least hearing loss ear (B).

(iv) A midline value was then calculated by the subtraction of B from A, which value is then halved, and the result added to the B value. This is the TRP baseline.

(v) All of the dB HL thresholds from the audiogram were then added to the values in Table 2 above which is the summation of the ISO 40 Phon ELC correction values, and the dB HL to dB SPL transfer functions. This produces a measure of hearing in terms of the relative perceived loudness of stimuli at each of the discrete frequencies. The values were expressed in dB SPL so that the desired equalization frequency response could be determined within the 24 dB SPL range of the graphic equalizer.

(vi) The baseline value was then subtracted from each transformed threshold, and its result then multiplied by a 0.4 gain. This process is repeated for each frequency of each ear.

(vii) These values were then used to manually set the graphic equalizer with the left ear's required equalization response (REQ) used in the left channel, and the right ear's REQ used in the right channel of the equalizer.

The audiogram for the participant chosen to demonstrate how the TP1 accounts for a steeply-sloping asymmetrical hearing loss (see FIG. 3), was also chosen to demonstrate how the TRP algorithm modifies the intensity of the audio signal at selected frequencies to provide relief of the perception of the tinnitus. Tables 3 and 4 below show the calculations at each frequency for the left and right ears respectively using the TRP algorithm above. The baseline calculation was made as follows:

$$\begin{matrix}Baseline = 0.5 ( A - B ) + B \\ [ 0.5 ( L SPL 10 + L SPL 12 ) - 0.5 ( R SPL 0.5 + \\ R SPL 0.75 ] \times 0.5 + 0.5 ( R SPL 0.5 + R SPL 0.75 ) \\ = [ 0.5 ( 89.5 + 91 ) - 0.5 ( 1.5 + 8.5 ) ] \times 0.5 + 0.5 ( 1.5 + 8.5 ) \\ = ( 90.25 - 5 ) \times 0.5 + 5 \\ = 47.625\end{matrix}$$

TABLE 3

| | | | Corrections and Calculations | | | | | |
|---|---|---|---|---|---|---|---|---|
| | | | ELC | | | | | |
| | | | & SPL | | | | | |
| L. | P's | | transfer | | | | | |
| Freq | dB | P's | func- | | − | | | |
| (Hz) | HL | SPL | tions | = | Baseline | = | ×0.4 | =REQ |
| 250 | 5 | | 23.5 | 28.5 | 47.625 | −19.15 | ×0.4 | −7.66 |
| 500 | −10 | | 7.5 | −2.5 | 47.625 | −50.15 | ×0.4 | −20.06 |
| 750 | 2.5 | | 5.5 | 8 | 47.625 | −39.65 | ×0.4 | −15.86 |
| 1000 | 15 | | 7 | 22 | 47.625 | −25.65 | ×0.4 | −10.26 |
| 1500 | 25 | | 6.5 | 31.5 | 47.625 | −16.15 | ×0.4 | −6.46 |
| 2000 | 40 | | 7 | 47 | 47.625 | −0.65 | ×0.4 | −0.26 |
| 3000 | 65 | | 5.5 | 70.5 | 47.625 | 22.85 | ×0.4 | 9.14 |
| 4000 | 60 | | 2.5 | 62.5 | 47.625 | 14.85 | ×0.4 | 5.94 |
| 6000 | 60 | | 16.5 | 76.5 | 47.625 | 28.85 | ×0.4 | 11.54 |
| 8000 | 60 | | 21 | 81 | 47.625 | 33.35 | ×0.4 | 13.34 |
| 10000 | 80 | 89.5 | 16.5 | 96.5 | 47.625 | 48.85 | ×0.4 | 19.54 |
| 12000 | 80 | 91 | 13 | 93 | 47.625 | 45.35 | ×0.4 | 18.14 |

TABLE 4

| | | | Corrections and Calculations | | | | | |
|---|---|---|---|---|---|---|---|---|
| | | | ELC | | | | | |
| | | | & SPL | | | | | |
| L. | P's | | transfer | | | | | |
| Freq | dB | P's | func- | | − | | | |
| (Hz) | HL | SPL | tions | = | Baseline | = | ×0.4 | =REQ |
| 250 | 20 | | 23.5 | 43.5 | 47.625 | −4.15 | ×0.4 | −1.66 |
| 500 | −10 | 1.5 | 7.5 | −2.5 | 47.625 | −50.15 | ×0.4 | −20.66 |
| 750 | 0 | 8.5 | 5.5 | 5.5 | 47.625 | −40.12 | ×0.4 | −16.85 |
| 1000 | 5 | | 7 | 12 | 47.625 | −35.65 | ×0.4 | −14.26 |
| 1500 | 0 | | 6.5 | 6.5 | 47.625 | −41.15 | ×0.4 | −16.46 |
| 2000 | 15 | | 7 | 22 | 47.625 | −25.65 | ×0.4 | −10.26 |
| 3000 | 45 | | 5.5 | 50.5 | 47.625 | 2.85 | ×0.4 | 1.14 |
| 4000 | 30 | | 2.5 | 32.5 | 47.625 | −15.15 | ×0.4 | −6.06 |
| 6000 | 30 | | 16.5 | 46.5 | 47.625 | −1.15 | ×0.4 | −0.46 |
| 8000 | 20 | | 21 | 41 | 47.625 | −6.65 | ×0.4 | −2.66 |
| 10000 | 60 | | 16.5 | 76.5 | 47.625 | 28.85 | ×0.4 | 11.54 |
| 12000 | 75 | | 13 | 88 | 47.625 | 40.35 | ×0.4 | 16.14 |

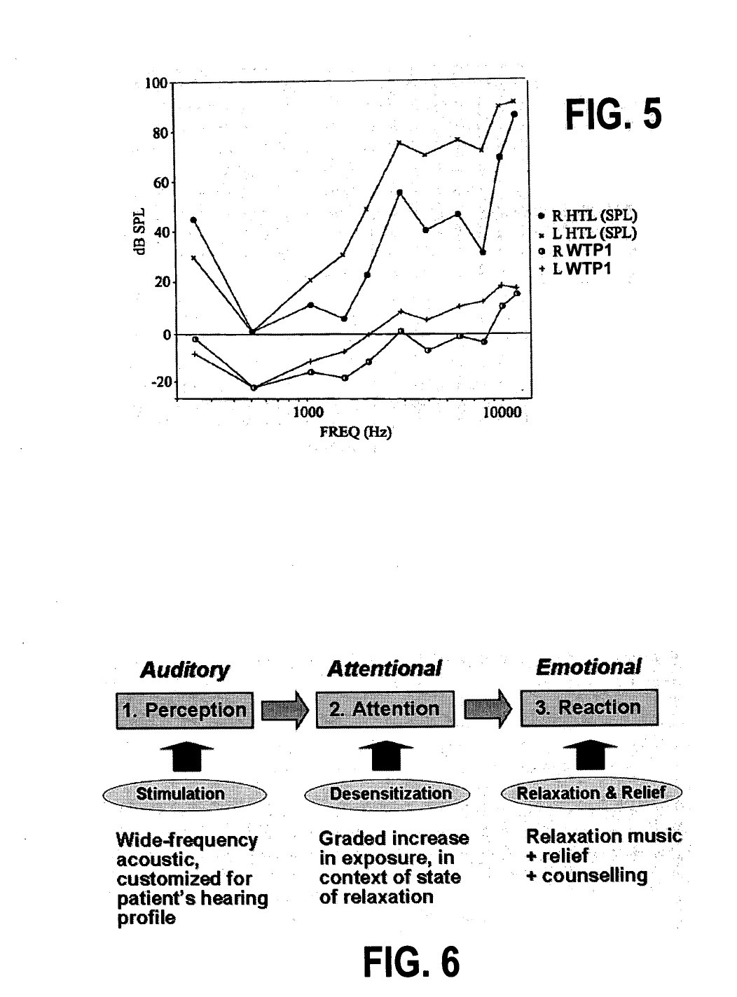

The REQ equalization curves for both ears are illustrated graphically in FIG. 5. A comparison of FIG. 5 with FIG. 3 will confirm that the patient's right and left hearing thresholds [HTL (SPL)] curves are identical.

In view of the results of the first clinical study and the additional understanding of the inventors, a second clinical study was conducted in which 90 people who suffer from tinnitus participated. The participants were allocated into one of four treatment groups: one group to test a second tinnitus protocol (TP2) which was intended to provide substantially complete masking, i.e., a high degree of relief and interaction, one to test the tinnitus retraining algorithm (TRP) which was intended to provide intermittent masking/relief/interaction, one to empirically measure the current TRT approach of using low-level broadband noise stimulation, and a quasi-control group to receive counseling alone. The second study exceeded expectations, with materially improved levels of habituation experienced by more than three-quarters of the participants using spectrally modified music. The adoption of bibliotherapy and TRT-style counseling resulted in significant improvements in clinical outcomes for all treatment groups. However, counseling alone appeared to be insufficient treatment for most participants. An important finding was that the TRP group experienced the greatest mean improvements in tinnitus distress. The TP2 stimulus group initially displayed a more rapid improvement, but the more gradual gains of the TRP group were sustained for longer, and ultimately were superior. There was little difference between the noise and counseling alone groups at post therapy and follow-up, although the mean improvements experienced by the counseling alone group were ultimately not statistically significant. While all treatment groups recorded mean reductions in tinnitus distress over therapy, the two music groups ultimately appeared to be the most effective. Approximately three-quarters of the music group participants experienced significant habituation to their tinnitus (TP2=78.6%, TRP=75%).

There were substantial reductions in ratings of reduced sound tolerance (e.g., hyperacusis) for both music groups, and a slight reduction for the noise group. The group without acoustic stimulation (counseling-only) displayed deterioration in reduced sound tolerance ratings over the same period, strongly indicating that the provision of acoustic stimulation was a key ingredient in the reduced sound tolerance improvements. The music group participants often reported that their reduced sound tolerance levels tended to improve faster than their tinnitus perception.

The clinical studies therefore suggest that total masking with music is more effective to facilitate a rapid improvement in distress and relaxation levels, despite the fact that intermittent masking/interaction with music eventually proved to be more effective on several measures. This indicates that a two-stage approach might be most efficient, whereby patients initially should employ a total masking algorithm to give a stronger sense of relief and control, then later switch to intermittent masking/interaction to remove the tinnitus detection. Maximizing the sense of relief and control in the early stage of the treatment is desirable for many patients who may be in a severe state of distress prior to commencing treatment for their tinnitus. Following a period of listening to the stimulus in a manner which facilitates relief and control, such patients may then proceed more comfortably into a treatment phase in which the tinnitus perception is exposed more fully in order to promote habituation or desensitization. Although the present disclosure is not to be limited by the following theory, it is believed that such a phased approach may facilitate habituation or desensitization to the tinnitus by providing the patient with a form of systematic desensitization. Within the context of a relaxation stimulus (i.e. relaxing music), the patient is presented in the successive phases as described above with a graded increase in exposure to the tinnitus perception during listening sessions. It is believed that this assists with retraining of the neurological processes relating to attentional focus on the tinnitus perception and the patient's emotional reaction to it.

Although the present disclosure is not to be limited by the following theory, it is believed that there are interrelated processes in the development of disturbing tinnitus (each of which is believed to involve at some level neuro-plastic changes in the brain). As illustrated in FIG. 6, these processes may be characterized as involving (1) changes within the auditory system which lead to the initial perception of the tinnitus sound, (2) the attentional filters in the brain which cause the patient to pay attention to the tinnitus perception, and (3) the emotional response and the autonomic nervous system which cause an adverse reaction to the tinnitus.

More specifically, with respect to initial tinnitus perception, it is believed that auditory deprivation causes the auditory system to become more active and more sensitive to sound. Following peripheral hearing damage, for example through noise insult or ototoxic drugs, there are changes in levels and nature of activity in the auditory nerves which appear to be centrally mediated. As a consequence, the auditory cortex receives altered neural input, which it interprets as sound. Essentially, it is believed that the cortex detects the amplified background neurological activity, and interprets it as the sounds perceived in tinnitus. These changes in the auditory cortex may also involve reorganization of the tonotopic map. With respect to the awareness of tinnitus, it is believed that perceptual filters at work on all of the senses determine which sensory perceptions are brought to our conscious attention and which are not. These filters play an important function as they allow the brain to focus on what is important while preventing us from being overwhelmed by sensory input. These filters recognize specific patterns of neural activity, and are constantly being updated and refined through experience. In the case of tinnitus, an importance “label” is applied to the tinnitus sound, such that it is constantly brought to the patient's conscious attention. With respect to the emotional (limbic) and autonomic nervous system engagement, it is believed that the limbic system of the brain, which controls the patient's emotional state, and the autonomic nervous system, responsible for the so-called fight or flight reflex, become engaged in response to the awareness of tinnitus. This causes a stressful state of high arousal and anxiety in response to the tinnitus awareness, which has a significant impact on the quality of life and general well being. This reaction also reinforces the other two processes referred to above, i.e., it leads to further increases in the sensitivity of the auditory system, and reinforces the attentional filters. This in turn leads to further increase in tinnitus loudness and awareness, which in turn increases the level of stress, and so on, in a self-perpetuating cycle that can make the tinnitus progressively worse over time.

Accordingly, in view of this understanding, certain exemplary embodiments discussed throughout this disclosure address some or all of these understandings. For example, certain embodiments may deliver a broad frequency stimulus into the system to counter the need for increased auditory sensitivity due to auditory deprivation. The broad frequency stimulus may be spectrally modified to account for each patient's hearing loss profile and the modification may be performed separately for each ear and then the resultant stimuli may be combined in a manner that provides a balanced perception across the two ears, and is delivered in stereo to stimulate the integrative pathways of the auditory system as well as enhance the listening experience for the patient. In this way, the treatment stimulates as much of the system as possible, as evenly as possible, and thereby reduces the need for the brain to “turn up” the sensitivity in the auditory system.

Additionally, exemplary embodiments may use music, which aims to address the limbic system/autonomic nervous system involvement that causes the aversive reaction to tinnitus. This aspect draws on the belief that relaxation music is as effective as progressive muscle relaxation in generating a relaxation response and is further reinforced by the relief and sense of control that comes from being able to shut out the tinnitus sound as well as by improvements in sleep that may result. All of these factors may lead to a reduction in the level of limbic system arousal and the consequential stress response.

Furthermore, certain exemplary embodiments may address the attentional filters using the principles of systemic desensitization. That is, because of the dynamics of the music, once customized for the particular patient, the stimulus provides relief to the patient in the peaks of intensity in the music, while allowing the tinnitus to be momentarily perceived in the intensity troughs. By gradually increasing the degree of exposure to the tinnitus perception over time, the brain may be retrained to perceive the tinnitus sound but not to pay particular attention to it, and therefore, not to trigger a stress response to react to it.

Generally, and for reasons detailed throughout this disclosure, exemplary solutions may consist of at least 2 stages: a 1st Stage and a 2nd Stage. In both stages, the acoustic signal is provided with pleasing and relaxing sounds making the treatment easy and pleasant to use. The 1st Stage typically provides an acoustic signal with a high level of interaction with the auditory system disorder to provide relief while using the treatment. The 2nd Stage provides an acoustic signal with a lower level of interaction with the auditory system disorder. In clinical trials, the “intermittent” exposure of the effects of the auditory system disorder during the 2nd Stage creates a desensitization process, and has proven to be efficient and effective at reducing awareness of the auditory system disorder and the associated disturbance. In clinical practice, it has been the inventors' experience that more than two phases of treatment may be advantageous for some patients. For example, in the event of a deterioration in tinnitus as a result of stress, noise exposure or other exacerbating factors, some patients may find it desirable to temporarily transition from an intermittent interaction phase to once again utilize a high interaction phase in order to reemphasize the relief and control aspects of treatment.

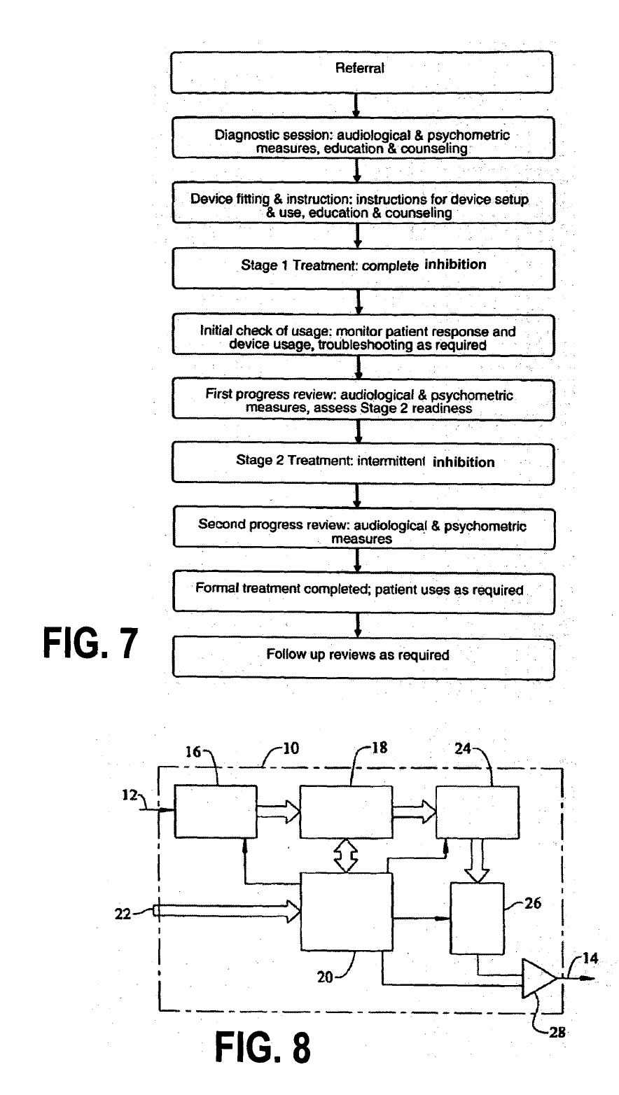

An embodiment of a 2 stage tinnitus rehabilitation method is outlined in FIG. 7. Following referral, for example from an Ear Nose and Throat specialist or other clinician, the process begins with diagnostic tests of the patient's audiological characteristics, as well as education as to the likely pathogenesis of their tinnitus. A tinnitus treatment device is then prescribed with embedded acoustic therapy customized for the patient (e.g., spectrally modified music plus added noise) and instructions provided for its use so as to provide complete masking (Stage 1 of the method). The patient's response is checked a short period (e.g., around two weeks) later and any difficulties that the patient may be encountering in using the device are discussed and resolved. After a further period of, for example, four to ten weeks (e.g., five, six, seven, eight, etc.), subject to patient readiness, the Stage 2 acoustic signal (e.g., spectrally modified music without added noise) is provided, with instructions for its use so as to provide intermittent masking/interaction and so greater exposure of the patient to their tinnitus. Progress review appointments include the measurement of key audiological and psychometric parameters in order to monitor progress and provide positive feedback to the patient. In addition, these appointments include a review of patient compliance, for which patient usage information has been logged and stored within the device for retrieval and review by the clinician. Throughout treatment, patients are instructed to adjust the volume setting on the device at the beginning of each listening session. During Stage 1 of the method, the volume is set so that the combined spectrally modified music/noise signal just masks the tinnitus. During Stage 2, the volume is set so that the tinnitus is masked during the musical peaks, and is momentarily apparent during the troughs; as patients become progressively more habituated to their tinnitus, the perceived level of tinnitus may decline over time, and accordingly, patients may need to set the volume progressively lower from session to session.

In the clinical studies, pre-recorded music was spectrally modified using the predetermined algorithms, and re-recorded on audio cassette tapes for participants' use. In light of copyright considerations, purchase of the rights to re-record music from selected recording companies or the commissioning of special purpose recordings may be required. In certain exemplary embodiments a programmable device for use by private practitioners may be provided. The device thus envisaged can be programmed by a qualified audiologist or health professional to account for each individual's tinnitus and hearing loss characteristics, using the tinnitus algorithms and clinical protocols described herein. In certain embodiments, the device may take the form of a musician's hearing aid-type device designed to spectrally modify the audio signal as it enters the wearer's ears. Other exemplary embodiments may provide the device in the form of a device which can be employed in conjunction with a PMP and has an input adapted to connect to the audio output headphone jack on the PMP. The device would have a standard headphone jack to which a conventional PMP headphone can be connected. In an alternative exemplary embodiment, a modified sound recording is automatically generated in the audiologist's clinic, tailored to the patient's audiometric configuration, using software accessed via the World Wide Web.

FIG. 8 illustrates in schematic block diagram form an exemplary embodiment of a tinnitus rehabilitation device. The device 10 has an input 12 adapted to receive a two-channel stereo signal from the headphone output jack of a PMP. The device 10 also has an output 14 which provides a two-channel stereo signal, spectrally modified by a predetermined algorithm programmed into the device 10, which is suitable for listening to through a conventional PMP headphone. In certain embodiments, the device 10 employs digital signal processing, and therefore the left and right input audio analog signal is converted to digital format in an analog to digital converter (ADC) 16. The digital output signal of ADC 16 is then sent to a digital filter 18 which filters the digitized audio signal in accordance with a predetermined algorithm. The digital filter 18 modifies the intensity of the audio signal at selected frequencies in accordance with the algorithm.

The filter characteristic of the digital filter 18 may be programmed manually using thumbwheels or a similar interface or the digital filter 18 may be programmed electronically by means of a microprocessor-based controller 20 having a communications port 22 that may be connected to a desk top computer. Using a custom-designed software program which may accompany the device 10, an audiologist or other hearing aid dispenser can program the device 10 by means of a graphic user interface (GUI) which facilitates the input of the required clinical data into the non-volatile memory of the controller 20. Thus, for example, the clinical audiologist may simply enter the patient's pure tone hearing level thresholds at each of the 10 discrete frequencies from 0.25 to 12 kHz. The audiologist may also be required to enter the two adjacent least hearing loss frequencies (B) the hearing threshold at the frequency of tinnitus pitch match (A) and/or the two adjacent greatest hearing loss frequencies (C). Either the software or the controller 20 will then use these figures to calculate the baseline value, and employ the predetermined algorithm to calculate the required equalization values. These values are employed by the controller 20 to set the filter constants at each frequency in the digital filter 18.

The device 10 may also include an additional signal processing means 24, which is also under control of the controller 20, for providing further spectral modification of the digital audio signal after filtering by the digital filter 18. The spectrally modified audio signal is then converted back to analog format in a digital to analog converter (DAC) 26. An amplifier 28 may be provided to control the amplitude of the analog output signal provided at the output 14 of the device. It will be understood that each of the digital components of the device 10 may be integrated into a single integrated circuit, so that the dimensions of the device 10 can be made quite small and the device therefore remains inconspicuous.

In certain embodiments, the proprietary algorithms or digital processing of the audio signal may be entirely software-based, facilitating the production of a stored music medium (tape or compact disc or alternative medium in an uncoded format, or using MP3, WMA or other coded format) for playback by the tinnitus sufferer on a standard personal sound reproduction system, such as a personal music player (PMP), with headphones. In this embodiment, the method of providing a tinnitus rehabilitation sound recording takes full advantage of the speed and economies provided by the Internet for fast digital communications and remote processing power. With no more than a desktop personal computer (PC) with CD-writing capability, the ability to provide a customized tinnitus rehabilitation sound recording can be placed at the fingertips of the audiologist, healthcare professional, or patient. By utilizing the reach of the World Wide Web and developing an application service provider (ASP), (also described as “on-line operating software”), the method can be extended to provide tinnitus relief and treatment to a global market.

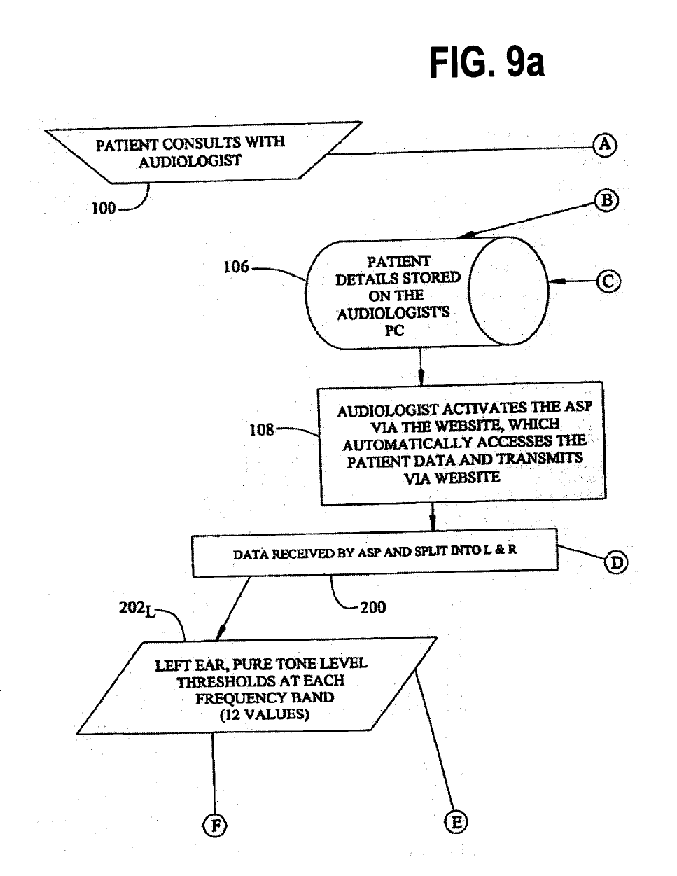

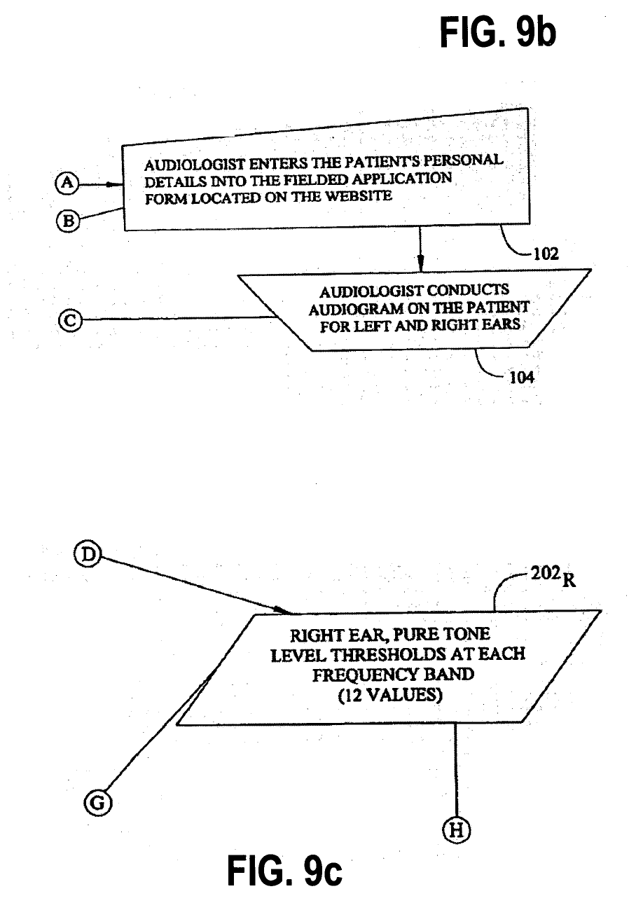

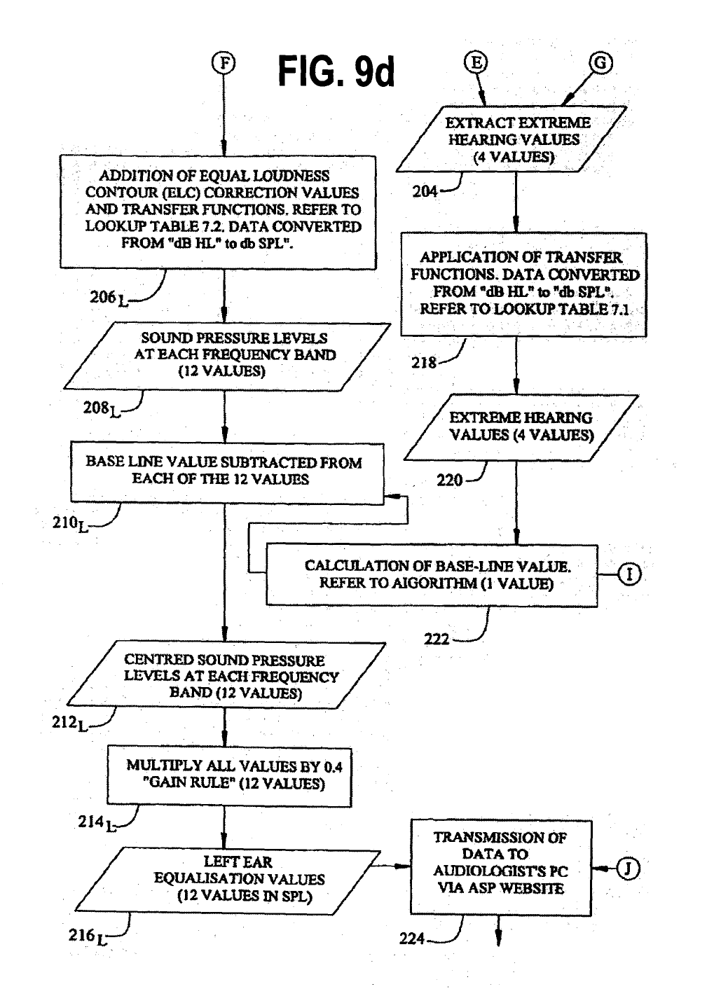

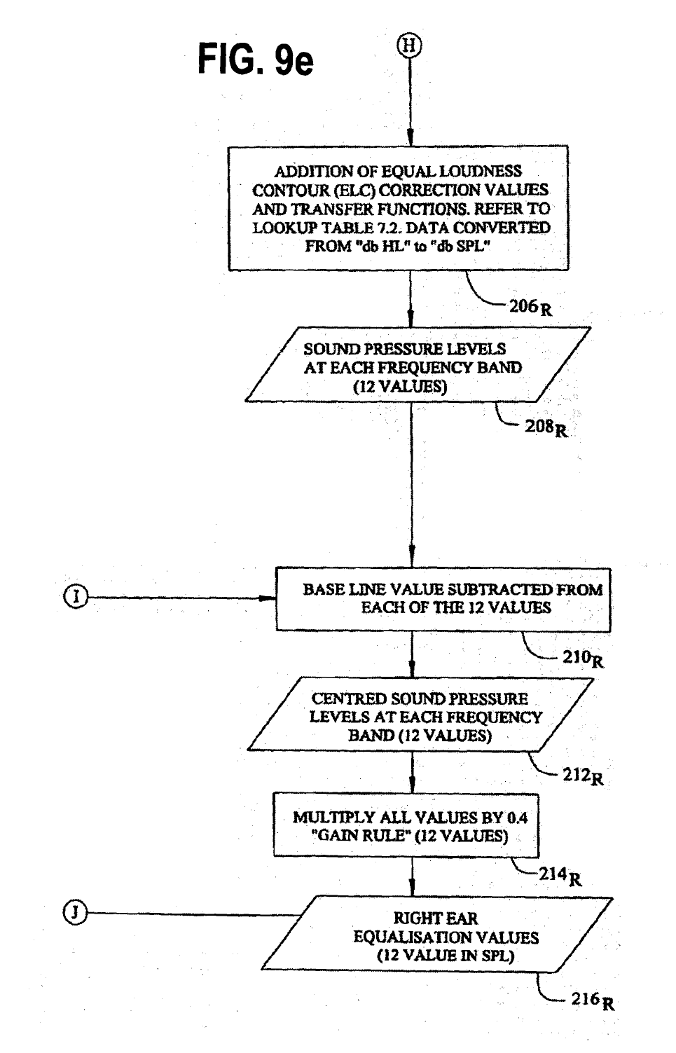

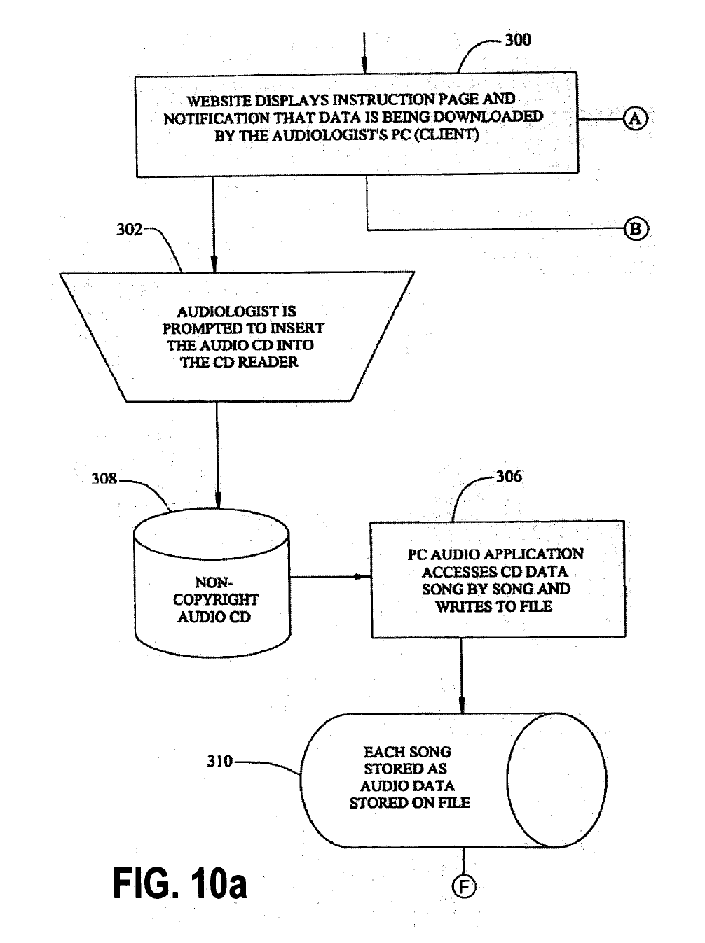

FIGS. 9 and 10 illustrate in flowchart form an exemplary method of providing a tinnitus rehabilitation sound recording utilizing the World Wide Web and the services of an ASP.

In this embodiment, the process commences in the audiologist's clinic (in other embodiments, the healthcare professional or audiologist may not be used) where the patient consults 100 with the audiologist. The audiologist enters 102 the patient's personal details into the appropriate fields in an application form located on a proprietary website. The audiologist then conducts 104 an audiogram on the patient's left and right ears. The audiogram is converted into an appropriate digital format and stored 106 on the audiologist's PC. The audiologist may then activate 108 the application service provider (ASP) via the website, which automatically accesses the patient data, including the digital audiogram, and transmits it via the website to the ASP.

Data is received 200 by the ASP and split into left and right ear processing channels. A central processing server (accessed via the ASP) houses the software containing the predetermined algorithms for converting the patient data to a digital filtering format herein referred to as a predetermined profile. This predetermined profile is then transmitted back to the audiologist's PC. The central processing server uses the digital audiogram to determine 202L, 202R the pure tone level thresholds at each of the predetermined frequencies for the left and right ears. The software ascertains 204 the two adjacent greatest hearing loss frequencies in the greatest hearing loss ear, and also the two adjacent least hearing loss frequencies in the least hearing loss ear. In each of steps 206L, 208L, 210L, 212L, 214L, 216L and 206R, 208R, 210R, 212R, 214R, 216R the tinnitus retraining protocol algorithm is applied to the left ear and right ear levels respectively, as is illustrated graphically in Tables 3 and 4 above.

In steps 218, 220 and 222 the baseline value is calculated, which is subtracted from each of the transformed threshold values for the left and right ears at 210L, 210R. The left and right ear Required Equalization Response (REQ) values are then transmitted 224 to the audiologist's PC via the ASP website. The website, which is visible on the audiologist's PC, notifies 226 the audiologist that the REQ values are being downloaded onto the audiologist's PC, and also prompts 302 the audiologist to insert a music CD (or any suitable media for storing the music or audio file such as (a DVD, memory card, etc.) into a CD player connected to the PC. The audiologist is also prompted 304 to insert a blank CD into the CD writer connected to his PC. It is to be understood that any suitable audio recording may be employed, preferably a music recording, stored on any suitable storage medium, such as a compact disc, audio cassette or MP3 card. Typically, the patient is offered a choice of music CD's, for which the appropriate copyright license fees have been paid, to be used as the base recording. An audio software application on the audiologist's PC accesses 306 the CD recording 308 and stores 310 the audio data to a file in the memory of the PC.

Proprietary software accessed by the ASP online reads the audio files stored in the PC and splits the signal into left and right stereo signals 320. Meanwhile, the REQ data received by the audiologist's PC is allocated 316 a channel reference (i.e. left channel data and right channel data 318). The software then converts 320 this left and right channel data into left and right predetermined profiles 322 respectively including interpolated values, between the twelve band frequencies, across the full frequency range up to, for example, 12 KHz. Software provided on the audiologist's PC accesses 324 and, using a (Fast Fourier Transform) FFT process (e.g., in a manner that would be readily understood by those experienced in signal processing in view of this disclosure), applies the predetermined profile to the right and left signals for the audio files in order to produce the left and right channels of the spectrally modified music signal 328. The modified audio files 328, one corresponding to each of the songs on the original music CD, are then utilized 330 by the CD Writer Software stored in the audiologist's PC, and are written to a blank CD 332.

The advantage of using an ASP and the audiologist's PC is that the amount of data transmitted and the processing power required by the server is in relative terms, very low. It is the processing of the audio signal that requires the bulk of the processing power. Via this model that power is housed in the PC of the audiologist instead of the server. Processing time would be negligible and therefore the entire process could be encompassed in the one patient visit.

Transmission is either via e-mail using a secure line with encryption or via a password-restricted web page; only qualified audiologists having access. Additional security measures such as ‘one-time-only-downloads’ or limiting the time the data is available on the website are also possible.

The consultation can easily be held in conjunction with a therapy session with the audiologist or healthcare professional, or as part of a coordinated therapy regime of on-going treatment. Certain embodiments may include the download of the proprietary software from the website and a royalty on each data download, i.e. for each CD made (not per patient, as each patient may wish to modify more than one CD). The Internet website could also provide a number of other services to assist in the relief of and treatment of tinnitus and hyperacusis. Thus, while music is one preferred embodiment, CDs can also be produced using noise, environmental sounds, pure tones, speech signals, or combinations thereof. It is also possible that parties could enter audiogram details without the help of an audiologist. When the audiologist or hearing aid dispenser does not have a CD burner, facility will be available for the CD to be produced at the ASP or other site, then posted to the clinic. As the data transmission speed of the Internet significantly increases, facility will be available for the processing of the audio signal to be performed within the ASP server if required.

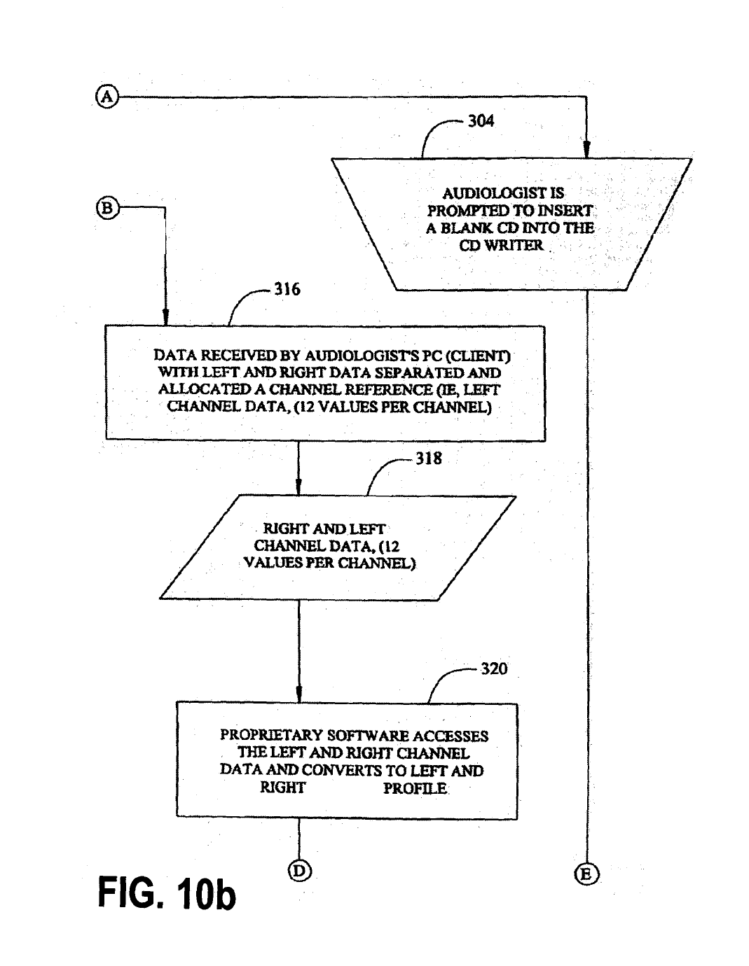

An exemplary embodiment of the tinnitus rehabilitation device 10 is shown in FIG. 11. The device 10 includes a stereo output 14 which may be used to connect the device 10 to headphones or earphones (not shown). Additionally, the device 10 includes a number of functional buttons for performing functions such as playing and stopping the audio signal, adjusting the volume of the audio signal and selecting whether to continue playing the same audio signal (replay function). Additional functions, such as those found on typical audio devices may also be added. As previously described, the device is in some embodiments small enough to be discrete and therefore, may include a battery. In certain embodiments, the battery may have a life that was sufficient to allow extended use (e.g., approximately one-week of use) without recharging. Also, as described above, the device may have a memory (e.g., a RAM card) for storing the audio signal such that an external media may not be required. In certain embodiments, a memory capable of storing sufficient audio signal to provide the patient with a choice of audio signals (e.g., approximately ½, 1, 2, 3, 4, 5, 6, hours or other time periods of listening time) may be used.

Additionally, a volume control may be provided such that the patient can set the volume of the audio signal to an appropriate level. In certain embodiments, the volume function would reset to a minimum value at the beginning of each treatment session. In this manner, the patient may be required to adjust the volume to either, in Stage 1 of the method, fully mask, or, in Stage 2 of the method, intermittently mask their tinnitus, the perceived level of which, as described above, may vary between sessions. This may be advantageous for ensuring that patients set the volume to the level that is most appropriate, substantially appropriate, or more appropriate at each listening session, rather than leaving the level as it was when previously used; however, in certain instances, it may be appropriate to leave the level as it was when previously used. For example, in some embodiments, the volume may be reset to a zero value or to a level that is just audible by the patient. In some embodiments, the processor may be configured to perform the volume resetting function. In some embodiments, the volume may be reset to a value that is determined by a patient's particular profile (e.g., the patient's audiogram).

In an embodiment of the device 10, the audio signal may be outputted from an internal storage medium, onto which the customized signal has been stored. In an alternative embodiment, the filtering means is incorporated within the device such that it acts ‘on the fly’ to modify any input signal to generate a customized output signal. In this embodiment, the device 10 may also include a safety locking mechanism which prevents the outputting of any signal that does not include a specific coding which denotes that it has been appropriately modified so as to be appropriate for use by that patient. In certain embodiments, the filtering means might be incorporated within the device but the device may preprocess the input signal which is stored in the device 10 to create and store the customized output signal or a part of the customized output signal. This configuration may be beneficial in embodiments where the processor within the device is not fast enough to act on the fly or where the battery life of the device was limited and processing would diminish the battery life prematurely.

Although many of the exemplary embodiments discussed herein use an audiologist to take a patient's full audiogram and use that patients data to create a profile which is used to produce the spectrally modified music signal, neither the audiogram or audiologist is required in certain embodiments.

In certain embodiments, a self administered audiogram approximation may be provided and the resulting data may be used to create a predetermined profile which is used to produce the spectrally modified music signal.

For example, in certain embodiments, tones or bands of noise or tone combinations may be provided to the patient and the patient may turn up volume of the tone until it is audible. In this embodiment, the patient may be provided with these tones in a clinical setting or in a non-clinical setting. If provided in a non-clinical setting, the tones may be provided to the patient over the internet or at a kiosk in a commercial environment or by the device. The user may interact with an interface provided and based on the user's selected volumes, the system may generate a predetermined profile for the patient and, in certain embodiments, provide the profile to the patient. Alternatively, the tones may be provided with random amplitudes and frequencies and the patient may simply be required to indicate whether the tone is audible or not. In certain embodiments, this type of a process may be more reliable than allowing a patient to select appropriate volumes since it may allow for the creation of a more accurate audiogram approximation. In some embodiments, ranges of tones or multiple tones may be provided simultaneously.

In other embodiments, a graphic equalizer interface may be provided to the patient to allow the patient to select his/her preference. In this embodiment, the equalization interface may be provided on the device itself or alternatively, may be provided over the internet, in a kiosk, or other suitable means.

In certain embodiments, once the audiogram approximation is obtained, the appropriate data may be provided to the patient to load onto the device (e.g., the PMP device) so that the device can create the modified signal “on the fly” or the data may be stored with a provider (e.g., an entity that provides services or devices related to auditory system disorder rehabilitation) so that the audio signals can be modified by the clinician, other healthcare professional, or retailer before providing a modified audio signal to the patient.

In certain embodiments, a set of standard/generic profiles may be provided and a selected profile could be used to create a predetermined profile which could be used to produce the spectrally modified music signal or, in certain embodiments, the selected profile may be used to directly create the modified audio signal. For example, in certain embodiments, the patient may be able to select one profile from a number of predefined profiles. This selection may occur at the device through a user interface or may be provided in a clinical or commercial setting, including over the internet. In certain embodiments it may be desirable to provide a predefined set of profiles that are accessible in a structured fashion. This may be desirable if, for example, there is an unmanageably large number of possible profiles or if it is desirable to select the profile for the patient instead of allowing the patient to arbitrarily select a profile. In this embodiment, the patient may select a profile from a number of profiles based on a defined interface or logical structure (e.g., a hierarchical tree or decision tree) for determining the best profile. For example, the interface might ask the patient to answer a series of questions or alternatively or in combination, may use one of the other methods described herein (e.g., providing random tones) to select an appropriate profile (e.g., in certain embodiments, the system may use one of a number of predetermined profiles based on the audiogram approximation discussed above).

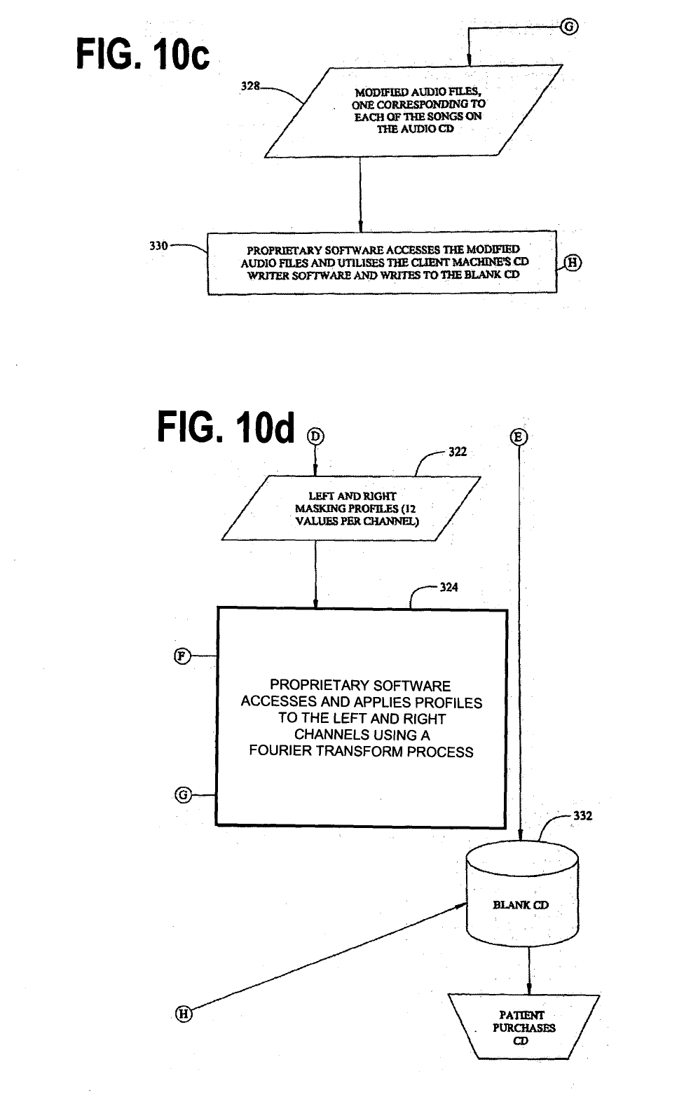

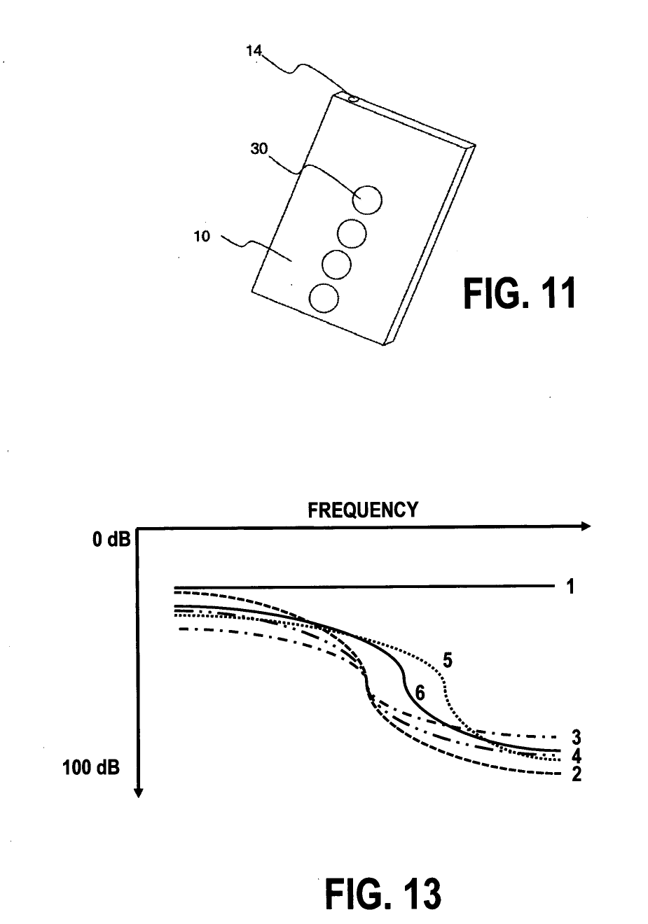

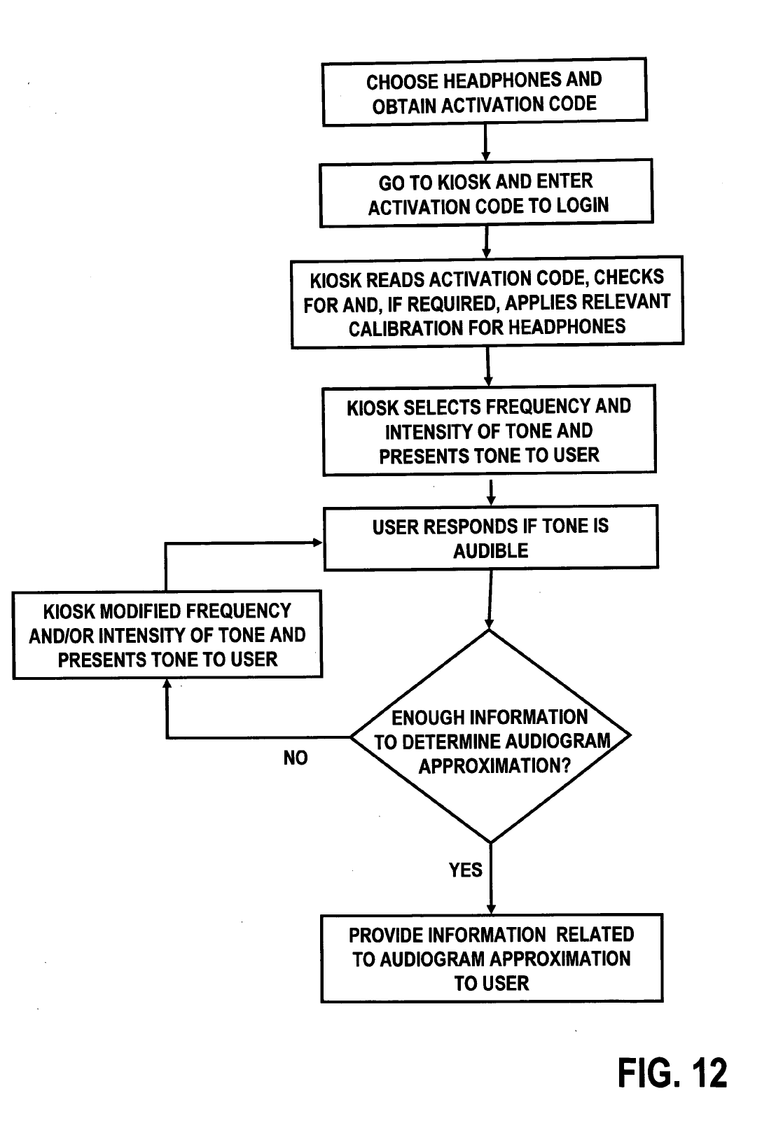

An embodiment for an exemplary method is illustrated in FIG. 12. As shown in FIG. 12, a user may arrive at a pharmacy or other desirable location and select headphones and obtain an activation code from a pharmacist or other third party. The activation code may include calibration information specific to the headphones in use. With the activation code and headphones, the user goes to a kiosk and logs into the kiosk to obtain a profile. The kiosk selects a particular frequency and/or intensity of a tone and presents the user with the tones via the headphones to individual ears and the patient responds when the tone is audible. This process is repeated until the kiosk has obtained enough information to generate a profile or audiogram approximation of the user. In certain embodiments, the kiosk may seek information at frequencies including, but not limited to, 0.25, 0.50, 1, 2, 3, 4, 6, 8, 10, and 12 kHz. The kiosk may select to test these frequencies by providing tones to the user at random or in a systematic manner. Alternatively, the kiosk may be programmed to zero in on the frequency of hearing loss by measuring the difference in thresholds and obtaining more measurements in the determined frequency range. For example, in certain embodiments, the kiosk may determine that there is hearing loss between 8 and 10 kHz and then seek further information at, for example 9 kHz; if there is no hearing loss at 9 kHz, the kiosk may then seek information about the users hearing threshold at 9.5 kHz.

Once the kiosk has the information required, it may provide the user with the necessary information for programming a corresponding device, or alternatively, may provide the information to a third party so that an employee can load the appropriate information onto the device.

In certain embodiments, the system may provide at least one profile that is adjustable by the patient or the clinician. The adjustment may be made using any combination of knobs, sliders, and or buttons on the device to adjust the low end and high end of the amplitude and/or the inflection frequency. The number of adjustments and/or the extent of the adjustment made can vary, and typically will vary, depending on the patient and/or the treatment being provided. In some aspects, the number of adjustments and/or the extent of the adjustments made may be at least 1, at least 2, at least 3, at least 4, at least 5, at least 6, at least 7, at least 8, or more depending on the particular circumstances. More specifically, and with reference to, for example, FIG. 13 (line 6), three adjustments could be made by a patient to adjust the modification provided to the audio signal. The patient may adjust the low end gain (left side of graph in FIG. 13), the high end gain (right side of FIG. 13), and the frequency at which the low end and high end are divided (i.e., the area frequency region in which the slope of hearing loss is steepest (e.g., the inflexion point)). The adjustments may be a continuous adjustment or may be incremental. Additionally, in some embodiments, the adjustment may be possible while the patient is listening to the audio signal. This type of an adjustment may be beneficial to modification prior to use in situations where the original audio signal is diverse (e.g., an audio signal from a television program, as discussed elsewhere herein).

Another embodiment of an exemplary method is described with reference to FIG. 13. In FIG. 13, the user is presented with multiple profiles and asked to select whether the currently presented profile is better or worse than the previously presented profile. In this embodiment, the selected profile may be used to modify the audio signal, that is, it may not be necessary for the system to obtain an audiogram or audiogram approximation. As shown in FIG. 13, the system presents the user with a first profile (1) (e.g., an audio signal modified by the first profile) as a base line and then presents a second profile (2) to the user and asks whether it is better or worse (based on the user's perception of relief (e.g., more or less relief) or some other benefit perceived by the user) than the baseline. Based on that response, the system provides a third profile (3) to the user and asks the same question of the user. By selecting various ratios of loudness between low and high frequencies and various frequencies associated with hearing loss, the system can identify one of a plurality of profiles that satisfies the needs of the user. As seen in FIG. 13, the difference between profile (2) and (3) is the loudness ratio and the same is the case of profile (3) and (4). Once the ratio is identified, profiles (4), (5) and (6) each adjust the frequency associated with the hearing loss.

As discussed, the audiogram, audiogram approximation, and/or predetermined profile could be generated independent of the device (e.g., clinic or at a commercial location) and a predetermined profile could be provided in a format that was appropriate for a number of devices (e.g., an existing PMP device). In certain embodiments, this may be distributed to the user as a software application (possibly over the web). For example, the software could be installed onto a patient's personal computer and used to generate the audiogram approximation or select a predetermined profile. Once this information is obtained, the software may also modify the audio signals to create the modified audio signals based on the predetermined profile so that the modified audio signal could be loaded onto a commercially PMP device.

In certain embodiments, such as the exemplary embodiments where the patient is presented with tones, it may be beneficial for the patient to utilize the same headphones as those that will be used during treatment. This allows the patient to achieve an appropriate adjustment for the headphones (e.g., a type of calibration). This may be beneficial since headphone frequency response can vary and optimal results using one set of headphones may not be optimal for another set of headphones. Therefore, by utilizing the same headphones when obtaining patient data and during treatment helps to ensure that the treatment is as optimal as possible. As discussed elsewhere herein, it may also be beneficial to include some headphone calibration information in the computation of the patients hearing loss (for example, when the user purchases the headphones an activation code and calibration information may be provide to the user).

As described throughout this disclosure, in certain embodiments, the acoustic signal may be a music signal. Music signals generally cover a wider spectrum of frequencies and are often considered enjoyable to listen but other types of sounds may be used as well as combinations of different audio signals. The signal may be spectrally modified in a clinical setting. If the clinic was to use music signals, the clinic may be able to record several hours of spectrally modified signals but it is likely that whatever the amount was, the listening experience by the individual would begin to be repetitive. For several reasons, it may not be possible for the clinic to create the necessary diversity that individuals may be seeking.

Generally, in certain embodiments, it may be desirable to have an acoustic signal that has a broad frequency, is relaxing enough but rich and distracting enough to reduce perception, has a dynamic intensity over time, is pleasant enough to listen to, and is customizable. Accordingly, in certain embodiments, acoustic signals from the television, radio, etc. might be used. It is also possible that the addition of video may create additional relaxation and distraction for the patient and may also improve compliance with the treatment procedure in certain embodiments.

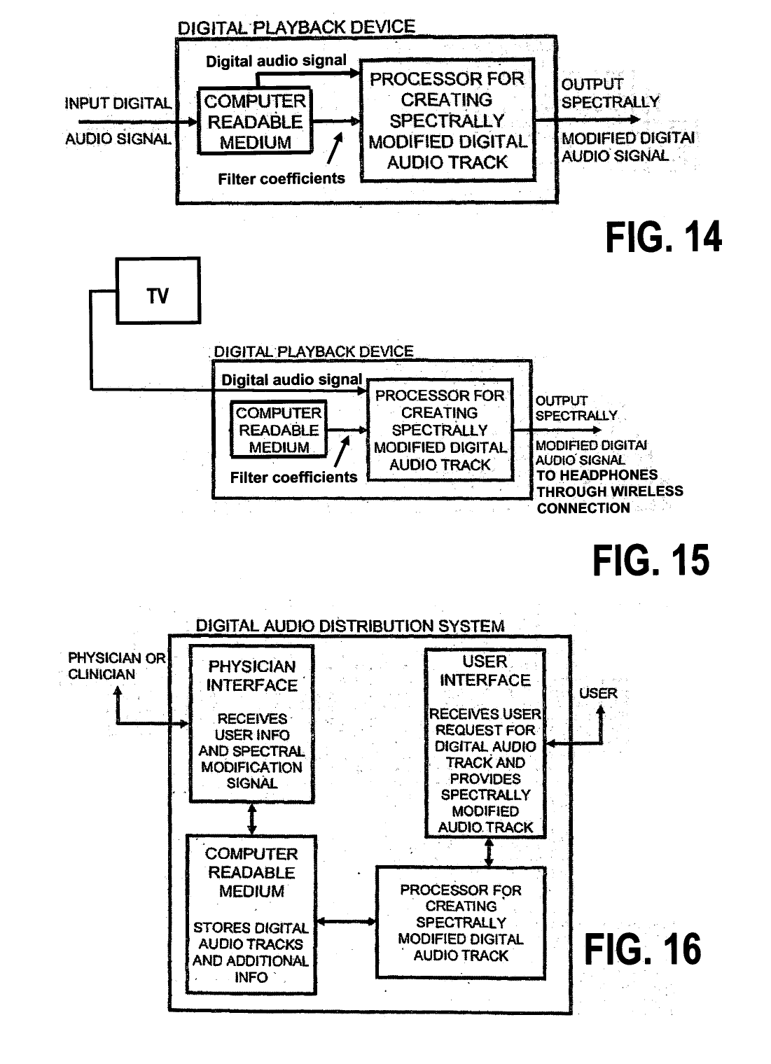

FIG. 14 is a functional schematic of a digital playback device in accordance with an embodiment of the present invention. As illustrated in FIG. 14, the digital playback device includes an input, a memory, a processor, and an output. The digital playback device may be a specialized device or in certain embodiments, the digital playback device may be a commercially available device such as an iPod (MP3 player), or some other device, such as for example, a set top box for a television. Generally, commercially available devices may include players of any format. Some common audio formats, for example, include, but are not limited to, MP3, WMA, WAV, MP2, RA, MPEG, and many other equivalents. It should be readily understood by a person of ordinary skill in the art that the present invention should not be limited to any specific set of file formats. These commercially available devices have become common because of their versatility, ease of use and inexpensive nature. In certain embodiments, the digital playback device may include an input for inputting an audio signal, a computer readable medium for storing the audio signal, a processor for receiving the audio signal and modifying the filtering coefficients (also referred to as a spectral modification signal and it should be readily understood that an actual signal such as an audio signal may be used or alternatively, an algorithm may be used to modify the digital audio signal and that regardless of whether the signal or the algorithm is used, the digital audio signal can be modified independently for the left and right channels, as discussed previously) used in the digital playback device to produce a spectrally modified audio signal; and an output for outputting the spectrally modified audio signal. In this embodiment, the audio signal is stored in its original format and then modified by the processor before playback (e.g., in real time or substantially real time). In some embodiments, the modified signal may also be stored in the digital playback device. Additionally, the modification of the filtering coefficients can be performed in numerous ways to reduce the effects of an auditory system disorder and/or to make the digital audio signal more enjoyable for an individual with or without an auditory system disorder. Specifically, instructions to modify the signal can be programmed into the processor from the factory, it can be loaded into the processor by a user or a clinic as software or firmware, or it can be implemented in hardware. In some embodiments, it may be adjustable by the user with controls on the device.

For example, and as discussed elsewhere in more detail, if the program is loaded from the factory, there may be different types of devices available. In one embodiment, for example, there may be two “models” for a device, a first model for spectrally modifying the audio signal for an individual with hearing loss at higher frequencies and increased sensitivity of loud noises at lower frequencies (See, e.g., FIG. 2) and a second model for modifying a signal for an individual with hearing loss at lower frequencies and sensitivity at higher frequencies. Of course, this is only an example and it should be readily understood that many different implementations could be effective, such as any of the exemplary embodiments for creating an audiogram, an audiogram approximation, or the use of a predetermined profile. In some embodiments, the digital playback device may also have a decoder for decoding the digital audio signal. For example, many commercial devices are able to play MP3, WMA, or equivalent files. In this case, the files are encoded in a specific manner and a special decoding device is generally utilized. If the decoding processor is provided, it is contemplated that the processor for spectrally modifying the digital audio signal may be incorporated in the decoding process or it may be provided separately. In situations where the processor is separate, the processor may be internal to the digital playback device or, in certain embodiments, may be coupled to the device externally. If the processor is coupled to the digital playback device externally, it may for example be coupled to the output of the digital playback device so that the processor can spectrally modify the digital audio signal before the spectrally modified digital audio signal is delivered to the user. In fact, an externally coupled device may allow the processor to be more readily programmed for the individual.

In certain embodiments, the device may include a compliance monitor for allowing a user to monitor how much time the user has used the device since it is generally important to use the device for predetermined amounts of time to be most effective. As with many digital playback devices, a battery for supplying power to the device is generally provided with sufficient battery life to allow extended use of the digital playback device without recharging. In some embodiments, the battery life may be at least 4 hours or as much as one week of regular use.

Additionally, since the digital storage technology improves daily, in certain embodiments, the computer readable medium for the digital playback device may be large enough to provide a diversity of audio signals. In certain embodiments, the computer readable medium storage capacity may be approximately equivalent to about 4 hours of the treatment signal or approximately 250 megabytes of capacity. Of course other capacities may also be desirable, for example, some device may have 20, 30, 40, or 60 gigabytes of capacity and sometimes even more while other devices may have as little as 10 megabytes of capacity or even less. The present inventions do not have to be limited to any specific capacity or capacity range.

In addition to playback, in certain embodiments, the digital playback device may include other advanced features. For example, since it may be important in certain embodiments to restrict the spectrally modified music to the individual for which the digital audio signal was modified, the digital playback device may include a user identification code in order to allow correct identification of the individual's own digital playback device in the event that more than one digital playback device gets placed together. Alternatively, so that the device may be shared, an on/off type of a switch may be used so that the digital playback device can be used without spectral modification. In another embodiment, the device may include a more advanced switch capable of spectrally modifying a digital audio signal in several different ways to accommodate several different individuals that may be sharing a device.

In certain embodiments, the digital playback device may also include a data downloading function for downloading logged information from the user device. Some information that may be useful, based on experience, is the times the device was used, the volume level of the device, what audio signals the individual listened to, etc.

The disclosed embodiments may be implemented in several ways. For example, the input on the digital playback device or the data downloading function described herein may be performed by a number of wired interfaces, infrared interfaces, or wireless interfaces.

In certain embodiments, the digital playback device may include a microphone as its input or as an auxiliary input. In this embodiment, addition of a microphone may be especially useful in for example, a theater or for television, radio or similar setting. The signal input from the microphone may be stored on the computer readable medium or it may be processed and output to a user without storage. In either situation, the spectral modification in “real time” or substantially “real time” situations may add additional benefit to individuals with auditory system disorders that they may not otherwise have. Such a feature may be an addition to a conventional/commercial device such as an MP3 player or it may be part of a purpose built device.

In certain embodiments, the digital playback device with a microphone or other analog or digital input may be used in conjunction with a television or other audio visual source (which may provide a higher compliance rate than other methods). In this embodiment, the device may be similar to the device illustrated in FIG. 2, in that the device may include an input, a processor for creating a spectrally modified audio signal and an output for outputting the spectrally modified audio signal. The input may include any suitable input for receiving an audio signal from a television (e.g., any analog or digital input). Some exemplary inputs may include, for example, a microphone, a stereo wired input (e.g., RCA connectors), or a wireless input, such as a Bluetooth wireless connection or the like. The device may also include a computer readable medium, but such a medium may not be required since, in certain embodiments, it may be desirable to process the incoming audio in a substantially “real time’ basis. The processor could be any suitable processor for spectrally modifying the audio signal such as any of the embodiments discussed throughout this disclosure.

For example, the processor could be implemented in software or in hardware as an infinite impulse response (IIR) filter or a finite impulse response (FIR) filter. The IIR filter may be for example, a 5^{th} or 6^{th} order filter and the FIR filter may perform a convolution covering, for example, between 24 and 256 samples depending on the complexity of the response required (e.g., 24 samples, 48 samples, 50 samples, 55 samples, 64 samples, 100 samples, 128 samples, 256 samples, etc). Additionally there may be one or more filters per channel and the accuracy of the filter may be between ±2-±20 dB (e.g., ±3 dB, ±6 dB, ±10 db, ±15 dB, ±18 dB, etc.). The filter parameters (or audiogram for, calculating the filter parameters) may be, for example loaded onto the device in a RAM or ROM type memory which may be integral with the device or may be removable (e.g., a card or similar device). In embodiments where music is loaded into the device with a removable memory card, it may be beneficial to load the filter parameters on the same card. The filter parameters may be automatically downloaded from the card to the processor to facilitate use of the processor with music or other stimulus from other sources. Additionally, in embodiments where a removable card is used, it may be possible to use the card as a user profile such that it could be inserted into a number of devices (e.g., a PMP and a set top box).

In some embodiments, the television audio may be combined with noise (e.g., broad band noise, white noise or substantially white noise) to further modify the input signal.

Since the television audio signal may be less predictable than for example, music (e.g., music distributed by a clinician), in certain embodiments, it may be beneficial to provide additional modification to the television audio (or, in certain embodiments, other audio such as patient selected music). Examples of other types of modification may include, for example, compression of taller intensity peaks and/or, partial modification if part of the audio signal is not conducive to appropriate modification.

In certain embodiments, the device may be a personal and portable device as discussed throughout this disclosure. In certain embodiments, the device may be a set top box that is coupled to the television, receiver, or radio. The set top box, may include a wired or wireless output for connecting headphones (as described above) to the set top box (alternatively, the set top box may be an intermediate component that is coupled between a series of devices and the connection to speakers and/or headphones may be with the television or other device). Additionally, in certain embodiments, the device may be integral with the television or associated component or may be implemented in software or firmware and loaded onto the television or associated component. In addition to other advantages described herein, embodiments in which the device is coupled to an audio or audio visual device such as a television or radio provide an additional benefit in that usage of the device for relief from the auditory system disorder can be more easily integrated into the user's daily activities (which may ordinarily include periods of listening to, for example, radio or television). In this way, compliance to treatment is facilitated, and hence potential for benefit from treatment further enhanced. In certain embodiments, the device may apply somewhat different factors and/or algorithms when modifying audio signals which are derived from different sources in order to account for the different properties of those signals. For example, it may be desirable to apply a different ‘M’ factor or different levels of attention (eg. by compression) of intensity peaks when processing an audio signal from a TV, radio or other ‘non-controlled’ source in order to reduce the risk that the patient is exposed to uncomfortably loud intensity peaks or other unpleasant audio transients.

An embodiment of an exemplary system is illustrated in FIG. 15. As seen in FIG. 15, the device is connected to the television via an appropriate wired connection and then a wireless connection is established between the device and a set of headphones. Of course, as would be readily understood, other connection types (such as those discussed throughout this disclosure) may be utilized. In this embodiment, it may be preferable for the device to be wirelessly connected to the headphones so that the device does not have to be located with the user. Additionally, as illustrated, the device includes an input for accepting a television audio signal, a computer readable medium (e.g., a memory card for storing the predetermined profile, filter coefficients and/or any noise that may be added to the audio signal), a processor and an output, each of which may be similar to those described with respect to FIG. 14.

In some embodiments, it may be important to distinguish between approved and non-approved signals. Specifically, depending on an individual's auditory system disorder, certain digital audio signals may not be amenable to spectral modification or may not be amenable to spectral modification beyond a certain extent. In these situations, the processor may be configured to distinguish between the two signals and therefore spectrally modify the signals to different extents. In some embodiments, the processor may not even spectrally modify a non-approved signal. For the processor to make such a determination, the processor may be configured to read the entire signal or may read a particular code recorded on the signal at some time prior to being used by the individual.

FIG. 16 is a functional schematic of a digital audio distribution system in accordance with an embodiment of the present invention. This embodiment focuses on the method for delivering digital audio signals to an individual or an individual's digital playback device. The digital playback device discussed with reference to these embodiments may be the same as the digital playback devices discussed herein or may be commercial off the shelf playback devices.

Generally, the system in accordance with certain embodiments utilizes a collection of digital audio tracks. One popular example of such a database is www.iTunes.com. This internet interface allows individuals to purchase digital audio tracks (or videos) individually and download them directly (or indirectly) to their digital playback device. Because this collection, and others as well, are stationary, it is possible for these databases to contain millions of audio tracks for an individual to select from.

In certain embodiments, a similar collection may be utilized or a new collection can be established. In either case, the individual may be able to access the system, and select at least one audio track. After selecting the audio track, the system can modify the digital audio track to create a spectrally modified digital audio track. The spectrally modified digital audio track is then provided to the user. In this manner, it may not be necessary for the digital playback device to include a processor for modifying the digital audio signal since it is modified before it is downloaded by the individual (although, as discussed above, it may be modified by the user as well). Additionally, although certain embodiments describe that the digital audio track is spectrally modified after it is selected, it is also contemplates a collection of spectrally modified digital audio tracks even though such a collection may be less desirable in certain situation given the versatility and storage space that would be required to store all of the spectrally modified digital audio tracks.

In certain embodiments, the individual may interact with the system over the internet or in some other acceptable means such as by visiting a store front or ordering over the phone.

As discussed, there are several ways that exemplary systems can determine how or with which spectral modification signal to modify the digital audio signal. In certain embodiments, the system may request user information to determine which of the predetermined number of spectral modification signals to use to create the spectrally modified digital audio signal. Specifically, as discussed above in more detail, the system may have a number of “generic” spectral modification signals and may pick one depending on certain criteria and information obtained from the individual.

In certain embodiments (also detailed herein), the system may allow the user to provide to the system a spectral modification signal to modify the at least one digital audio track. In this embodiment, the individual may obtain the spectral modification signal from a clinic or similar entity.

In certain embodiments, the user may select one of a predetermined number of spectral modification signals to modify the digital audio track and in some instances, the individual may obtain information from a clinic or similar entity or from a self administered test to determine which spectral modification signal to choose or the clinic or similar entity may indicate to the individual which signal to select. In a related embodiment, the clinic or similar entity may prescribe a certain spectral modification to an individual much like a drug prescription. Once the individual has the prescription, they will be able to provide the necessary information to the system such that the correct spectral modification signal is selected.

In yet another embodiment, the system may be able to gather enough information from an individual to create a customized or partially customized spectral modification signal. In this situation, the system may request information from the individual and may also administer certain tests to the individual such as an auditory test or the like and process the necessary data from the tests. In this manner, the individual may, in some embodiments, be able to obtain a customized spectral modification signal without visiting a specialized clinic, as described above with reference to exemplary embodiments.

As illustrated in the embodiment of FIG. 16, a physician interface is added so that the physician/clinician or entity creating the signals can directly interact with the system as well; in these embodiments, the physician/clinician or entity creating the signals may be able to provide spectral modifications to the system since the physician/clinician may be in the best position to determine how to produce generic signals. Alternatively, the physician/clinician or entity creating the signals could simply load the individual's specific spectral modification signal to the system and designate it as such. The user could then access the system and retrieve spectrally modified digital audio tracks based on their customized spectral modification signal. Additionally, by involving the physician/clinician or entity creating the signals, the system may also provide samples that could be provided to the individual.

Now that several embodiments of the auditory system disorder rehabilitation methods and devices have been described in detail, it will be apparent that the described methods and devices for providing relief for persons suffering from an auditory system disorder such as tinnitus and/or reduced sound tolerance has a number of significant advantages over prior art techniques, including, but not limited to, the following:
i) by facilitating the use of a personal music player with relaxing music, it is much more acceptable to patients than conventional devices, thereby promoting a pleasant listening experience, as well as compliance to treatment;
ii) by facilitating the use of a personal music player with relaxing music, facilitates relaxation, relief and a sense of control and hence ameliorates the limbic system reaction to tinnitus perception;
iii) by allowing the user to at least partially cover up their tinnitus perception, facilitates a sense of relief and control and hence ameliorates the limbic system reaction to tinnitus perception;
iv) the algorithms developed to spectrally modify the audio stimuli correct for each individual's particular hearing loss configuration as well as accounting for the effects of loudness recruitment, thus enabling effective stimulation at a relaxing intensity level, irrespective of each patient's audiometric profile;
v) it compensates for high frequency hearing loss which accompanies tinnitus in approximately 80% of cases, thus providing the broadest spectrum of acoustic stimulation;
vi) it compensates for each ear separately and delivers signals to each ear with control of the correlation between the two signals, thereby accounting for any asymmetry between the ears in order to facilitate stimulation at a comfortable listening level, as well as ensuring stimulation of the integrative pathways of the auditory system, and further enhancing the listening experience through provision of a stereo effect;
vii) intermittent tinnitus masking with music can provide a form of systematic desensitization to the disturbing effects of tinnitus; and,
viii) spectrally modified sound recordings produced using the algorithms reduce tinnitus distress to the point where it was no longer significantly interfering with quality of life in more than 75% of trial participants. Significant reductions in MMLs were measured, and levels of reduced sound tolerance had significantly improved.

It will also be apparent to persons skilled in the audiological and electronics arts that numerous variations and modifications may be made to the described method and device, in addition to those already described, without departing from the basic inventive concepts. For example, an algorithm may be employed to set the frequency response of existing tinnitus devices (e.g., maskers) which use bands of noise, rather than music, to achieve similar results. Various types of noise, pure tones and speech could also be used in addition to music. The same algorithms may also be employed in existing wireless receiver devices, such as the Starkey Silentia Set, or through hearing aid induction coil systems. Furthermore, the mathematical algorithms used for calculating the individual prescription of the audio signal may differ from the above-described algorithms, and extra sounds may also need to be inserted. However, other embodiments would be consistent with the clinical technique that is intended to provide a modification of the intensity of audio signals to account for hearing levels, specifically for the relief and/or treatment of auditory system disorders such as tinnitus and conditions of reduced sound tolerance (e.g. hyperacusis). All such variations and modifications are to be considered within the scope of the present inventions, the nature of which is to be determined from the foregoing description and the appended claims.

Many alterations and modifications of the present inventions will be comprehended by a person skilled in the art after having read the foregoing description. It is to be understood that the particular embodiments shown and described by way of illustration are in no way intended to be considered limiting. Therefore, references to details of particular embodiments are not intended to limit the scope of the claims, which in themselves recite only those features regarded as essential to the inventions.

The embodiments described herein are intended to be illustrative of the disclosed inventions. As will be recognized by those of ordinary skill in the art, various modifications and changes can be made to these embodiments and such variations and modifications would remain within the spirit and scope of the inventions defined in the appended claims and their equivalents. Additional advantages and modifications will readily occur to those of ordinary skill in the art. Therefore, the present disclosure in its broader aspects is not limited to the specific details and representative embodiments shown and described herein.

1. An audio playback device comprising:
a receiver configured to receive an audio signal;
a processor for spectrally modifying the audio signal in a substantially real time manner to compensate for an auditory system disorder; and
an output for outputting the spectrally modified audio signal to a person.

2. The device of claim 1, wherein the signal provides relief by providing at least one of: relaxation, stimulus to the auditory system, masking or partial masking, inhibition or partial inhibition, distraction, interaction with the person's tinnitus perception to a sufficient degree, reduction in the person's attentional focus on the tinnitus perception, reduction in the person's emotional reaction to the tinnitus perception, or an improved listening experience.

3. The device of claim 1, further comprising: a data storage device for storing data representing an audio profile of the person suffering from the auditory system disorder; wherein the processor is configured to process the audio profile data and combine it with the audio signal in a substantially real time manner to produce the spectrally modified audio signal.

4. The device of claim 3, wherein the audio profile is an audiogram.

5. The device of claim 3, wherein the audio profile is a self administered audiogram approximation that includes calibration information for headphones.

6. The device of claim 5, wherein the audiogram approximation is obtained by providing the patient with at least one tone and instructing the person to turn up the volume until the tone is audible.

7. The device of claim 5, wherein at least one tone is provided with a random amplitude and frequency and the person is instructed to indicate whether the tone is audible or not.

8. The device of claim 5, wherein the person is provided with graphic equalization functionality to select an appropriate equalization based on the person's preference.

9. The device of claim 3, wherein a standard or generic profile is selected from a set of standard or generic profiles.

10. The device of claim 9, wherein the person is instructed to select a profile from a number of profiles either by way of trial and error or by way of a defined interface or hierarchical tree structure for determining the best profile.

11. The device of claim 6, wherein a profile is selected from a number of predetermined profiles based on the audiogram approximation

12. The device of claim 3, wherein the profile is a single adjustable profile that can be adjusted to the person's preference.

13. The device of claim 1, wherein a masking algorithm provides intermittent masking of the tinnitus wherein, at a comfortable listening level, during peaks of the audio signal the tinnitus is substantially completely obscured, whereas during troughs the perception of the tinnitus occasionally emerges.

14. The method of claim 13, wherein the masking algorithm is of the form:
REQ=M(SPL+ELC(0.25,0.5,1,2,3,4,6,8,10,12 kHz)−Baseline)
Where:
REQ=Required equalization response of the Tinnitus Retraining Protocol
Baseline=0.5 (A−B)+B
A=mean dB SPL at the two adjacent greatest hearing loss frequencies in the greatest hearing loss ear
B=mean dB SPL at the two adjacent least hearing loss frequencies in the least hearing loss ear
SPL=hearing thresholds (in dB HL) converted to dB SPL
ELC=transfer values for 40 Phon Equal Loudness Contours
M=gain multiplier=0.3 to 0.95, and
Preferably M=0.4.

15. The device of claim 1, wherein the audio signal is associated with audio visual content.

16. The device of claim 1, wherein the audio signal is audio associated with a television broadcast.

17. The device of claim 1, wherein the audio signal is a highly dynamic signal whose spectral content and intensity constantly varies over time.

18. The device of claim 17, wherein the audio signal is a music signal.

19. A method for providing relief to a person experiencing tinnitus or conditions of reduced tolerance of loud sounds, the method comprising:
receiving an audio signal that is associated with a corresponding video signal;
processing the audio signal in real time so that the intensity of the audio signal is spectrally modified at selected frequencies; and
outputting the spectrally modified audio signal to a person,
wherein, in use, the combination of the video signal and the corresponding spectrally modified audio signal provide relief for an auditory system disorder.

20-32. (canceled)

33. A system comprising:
a user interface for obtaining data representing an audio profile of a user;
a receiver configured to receive an audio signal;
a processor for spectrally modifying the audio signal to compensate for an auditory system disorder;
and an output for outputting the spectrally modified audio signal to a person.

34-48. (canceled)
